(19) United States
(12) Patent Application Publication
(10) Pub. No.: US 2019/0127474 A1
(43) Pub. Date: May 2, 2019

(54) GALECTIN IMMUNOTHERAPY

(71) Applicant: THE COUNCIL OF THE QUEENSLAND INSTITUTE OF MEDICAL RESEARCH, Herston (AU)

(72) Inventor: Michelle WYKES, Brisbane (AU)

(21) Appl. No.: 16/226,302

(22) Filed: Dec. 19, 2018

Related U.S. Application Data
(63) Continuation of application No. 15/325,789, filed on Jan. 12, 2017, now abandoned.

(30) Foreign Application Priority Data
Jul. 14, 2014 (AU) 2014902709
Nov. 6, 2014 (AU) 2014904466

(51) Int. Cl. C07K 16/28; G01N 33/574; A61K 38/17; G01N 33/68

(52) U.S. Cl. CPC C07K 16/2851; G01N 33/574; A61K 38/1774; A61K 2039/505; C07K 16/2827; A61K 38/1732; G01N 33/68; C07K 16/2803

(57) ABSTRACT

A method of modulating immunity in a mammal is provided by modulating Galectin-9 activity in the mammal. Promoting or enhancing immunity may be effected by activating or stimulating Galectin-9 activity in the mammal, such as by administering a Galectin-9 agonist to the mammal. The agonist may be multimeric, soluble PD-L2 or an agonist antibody that binds Galectin-9. Suppressing or preventing immunity may be achieved by inhibiting or blocking Galectin-9 activity in the mammal, such as by administering an antagonist antibody or antibody fragment that binds Galectin-9 or that prevents or inhibits PD-L2 multimerisation and/or binding to Galectin-9. The aforementioned methods may be suitable for preventing or treating a disease, disorder or condition responsive to modulation of Galectin-9 activity. Also provided is a method of designing, screening, engineering or otherwise producing a Galectin-9 agonist, inhibitor or antagonist that may be useful for modulating immunity by modulating Galectin-9 activity.

### CROSS REFERENCE TO RELATED APPLICATIONS

This application is a continuation of U.S. patent application Ser. No. 15/325,789, filed Jan. 12, 2017, which is a national stage application under 35 U.S.C. 371 and claims the benefit of PCT Application No. PCT/AU2015/050393 having an international filing date of 14 Jul. 2015, which designated the United States, which PCT application claimed the benefit of Australian Patent Application No. 2014902709 filed 14 Jul. 2014, and Australian Patent Application No. 2014904466 filed 6 Nov. 2014, the disclosures of each of which are incorporated herein by reference.

### TECHNICAL FIELD

THIS INVENTION relates to immunotherapy. More particularly, this invention relates to targeting Galectin-9 to modulate the immune response and thereby prevent or treat one or more diseases, disorders or conditions.

### BACKGROUND

PD-1 is a member of the extended family of molecules that are known to down-regulate T cell function. PD-1 has two known ligands, PD-L1 (B7-H1) (Dong et al., 1999; Freeman et al., 2000) and PD-L2 (B7-DC) (Latchman et al., 2001; Tseng et al., 2001), which both belong to the B7 co-signalling molecule family. Expression of PD-1 can be observed on T cells, B cells, natural killer T cells, dendritic cells (DCs), and activated monocytes (Keir et al., 2008). PD-1 is not expressed on resting T cells but is inducible upon activation (Agata et al., 1996). Functional effects of PD-1 ligation can be observed within a few hours after T cell activation but PD-1 cell surface protein up-regulation requires 24 h (Chemnitz et al., 2004). When PD-1 is engaged simultaneously with the T cell receptor signals, it can trigger an inhibitory signal, although no signal transduction occurs when PD-1 is cross-linked alone (Sharpe et al., 2007). In general, interactions between PD-1 on T cells and its ligand, PD-L1, control the induction and maintenance of peripheral T-cell tolerance and negatively regulate proliferation and cytokine production by T cells during immune responses to pathogens or cancer (Sharpe et al., 2007). PD-L2 is another ligand for PD-1 which is generally thought to compete with PD-L1 for binding to PD-1. Generally, the immunological functions of PD-L2 appear to overlap with those of PD-L1 and there appears to be no specific role or function that can be attributed to PD-L2 per se.

### SUMMARY

The present invention has arisen, at least in part, from the unexpected discovery that Galectin-9 is a receptor for PD-L2. Accordingly, at least some of the immunological effects of PD-L2 may be mediated through binding of multimeric PD-L2 to Galectin-9 rather than through PD-1. The invention is therefore broadly directed to targeting Galectin-9 to thereby modulate the immune system. In one broad embodiment, the invention is directed to promoting or enhancing immunity in a mammal by activating or stimulating Galectin-9. In another broad embodiment, the invention is directed to suppressing or preventing immunity in a mammal by inhibiting or blocking Galectin-9.

An aspect of the invention provides a method of modulating immunity in a mammal including the step of modulating Galectin-9 activity in the mammal to thereby modulate immunity in the mammal.

A particular aspect of the invention provides a method of promoting or enhancing immunity in a mammal including the step of activating or stimulating Galectin-9 activity in the mammal to thereby stimulate or enhance immunity in the mammal.

Suitably, the method includes the step of administering a Galectin-9 agonist to the mammal to thereby activate or stimulate Galectin-9 activity in the mammal.

In one embodiment, the method includes the step of administering soluble PD-L2 or a biologically active fragment thereof to the mammal to thereby activate or stimulate Galectin-9 activity in the mammal.

Suitably, soluble PD-L2 is multimeric comprising n monomers wherein n≥3.

In one embodiment, the method includes the step of administering to the mammal an agonist antibody or antibody fragment that binds Galectin-9 to thereby activate or stimulate Galectin-9 activity in the mammal.

Suitably, this stimulates and/or initiates a Th1-mediated immune response and/or immunological memory.

Another particular aspect of the invention provides a method of at least partly suppressing or preventing immunity in a mammal including the step of at least partly inhibiting or blocking Galectin-9 activity in the mammal to thereby suppress or prevent immunity in the mammal.

Suitably, the method includes the step of administering a Galectin-9 inhibitor or antagonist to the mammal to thereby inhibit or block Galectin-9 activity in the mammal. Preferably, the inhibitor or antagonist at least partly prevents, or interferes with, a binding interaction between PD-L2 and Galectin-9.

In one embodiment, the method includes the step of administering soluble Galectin-9 or a biologically active fragment thereof to the mammal to thereby inhibit or block Galectin-9 activity in the mammal.

In one embodiment, the method includes the step of administering to the mammal an antagonist antibody or antibody fragment that binds Galectin-9 to thereby inhibit or block Galectin-9 activity in the mammal.

A related aspect of the invention provides a method of treating or preventing a disease, disorder or condition in a mammal including the step of modulating Galectin-9 activity in the mammal to thereby prevent or treat the disease or condition.

In one embodiment, the disease, disorder or condition is responsive to promoting or enhancing immunity by activating or stimulating Galectin-9 activity in the mammal. Preferably, the method includes the step of administering to the mammal an agonist antibody or antibody fragment that binds Galectin-9 to thereby activate or stimulate Galectin-9 activity in the mammal.

In another embodiment, the disease, disorder or condition is responsive to suppressing or preventing immunity by inhibiting or blocking Galectin-9 activity in the mammal. In one particular embodiment, the method includes the step of administering soluble Galectin-9 or a biologically active fragment thereof to the mammal to thereby inhibit or block Galectin-9 activity in the mammal. In another particular embodiment the method includes the step of administering to the mammal an antagonist antibody or antibody fragment that binds Galectin-9 to thereby inhibit or block Galectin-9 activity in the mammal.

Still yet another aspect of the invention provides a composition comprising a Galectin-9 agonist and an immunogen. Suitably, the composition is an immunogenic composition or vaccine that elicits an immune response to the immunogen. The immunogen may be a pathogen (e.g. an inactivated virus or attenuated bacterium) or a molecular component of the pathogen. Suitably, the composition comprises a suitable carrier, diluent or excipient.

A further aspect of the invention provides a method of designing, screening, engineering or otherwise producing a Galectin-9 agonist, inhibitor or antagonist, said method including the step of (i) determining whether a candidate molecule is an agonist which activates or stimulates Galectin-9 activity and is thereby capable of stimulating or enhancing immunity in a mammal; or (ii) determining whether a candidate molecule is an antagonist or inhibitor which blocks or inhibits Galectin-9 activity and is thereby capable of at least partly suppressing or preventing immunity in a mammal.

In one embodiment, in step (i) the candidate molecule mimics PD-L2 stimulation or activation of Galectin-9.

In one embodiment, in step (ii) the candidate molecule blocks or inhibits PD-L2 stimulation or activation of Galectin-9.

A still further aspect of the invention provides a Galectin-9 agonist, inhibitor or antagonist produced according to the method of the previous aspect.

A still yet further aspect of the invention provides a composition comprising a Galectin-9 agonist, inhibitor or antagonist of the previous aspect. Suitably, the composition comprises a suitable carrier, diluent or excipient.

Throughout this specification, unless otherwise indicated, “comprise”, “comprises” and “comprising” are used inclusively rather than exclusively, so that a stated integer or group of integers may include one or more other non-stated integers or groups of integers.

As used herein, indefinite articles such as “a” and “an” do not refer to or designate a single or singular element, but may refer to or designate one or a plurality of elements.

### BRIEF DESCRIPTION OF THE FIGURES

FIG. 1: PD-1 and PD-L1 modulate protective immunity against P. chabaudi and P. yoelii YM malaria. (a) Cohorts (n≥9) of WT and (b) PD-1KO mice were infected with non-lethal 10⁵ P. chabaudi or lethal 10⁴ P. yoelii YM pRBC and blood smears taken every 1-2 days to monitor parasitemia. After 40 days, all surviving mice were then rested for 140 days and re-challenged with the same parasite (arrow on x-scale). Error bars: ±S.E.M. Log scales highlight sub-clinical infections. All wild type mice died within 7 days of lethal challenge (†). (c) Total CD11c⁺ DCs from B6 WT (●) and PD-L1 KO (▴) mice infected with P. yoelii YM were transferred to naïve mice which were then infected with a lethal dose of P. yoelii YM, and mice examined every 1-3 days for >60 days. (Total n=9). All mice given WT DC died by day 9, while all mice given PD-L1KO DC survived.

FIG. 2: PD-L2 mRNA was compared with an average of 3 housekeeping genes by real time PCR in total spleen DCs from day 7 p.i. with P. yoelii YM and P yoelii 17XNL. Data are shown as the mean and range of mRNA levels obtained using RNA prepared in two independent experiments. Significance was analyzed using the non-parametric t-test on pooled data from replicate experiments.

FIG. 3: Blocking of PD-L2 in non-lethal infections exacerbates infection. Mean percent parasitemia in WT mice following treatment with control IgG (black circle), or blocking anti-PD-L2 antibody (white square). Data represent one of 2 independent experiments in WT (total n=10 mice) or PD-1 KO (total n=10) mice. (*p=0.0048).

FIG. 4: Soluble synthetic multimeric PD-L2 protects against lethal malaria and generates lasting immunity. Cohorts of B6 mice (n=12) were infected with lethal P. yoelii YM and on days 3, 5 and 7 mice were given either soluble octomeric PD-L2 or human IgG (Control Ig). After clearance of infection and resting for 3 months, the surviving mice were re-challenged with lethal P. yoelii YM infection again (arrow; no additional sPDL2).

FIG. 5: Soluble synthetic multimeric PD-L2 protects against symptoms of cerebral malaria and prolongs survival. Cohorts of B6 mice (n=9) were infected with P. berghei malaria which causes cerebral symptoms by day 8, and on days 3, 5 and 7 mice were given either soluble PD-L2 or human IgG (Control Ig). Mice were monitored daily for (a) cerebral symptoms and (b) survival. The mice ultimately died from absence of DC function due to excess TNF (Wykes, 2007).

FIG. 6: CD4⁺ and CD8⁺ T cell depletion studies show the role of these cells in protection against severe malaria. (a) Mean percentage survival of mice in WT mice following infection with lethal P. yoelii YM and treatment with rIg (black circle), or treatment with sPD-L2 (black square) with the depletion of CD4⁺ T cells (white square) or CD8⁺ T cells (white circle).

FIG. 7: Protection by sPD-L2 is not via blockade of PD-L1. Cohorts of WT and PD-L1 knockout mice were infected with lethal P. yoelii YM and treated with either Control IgG or sPD-L2. Mice were monitored for parasitemia.

FIG. 8: Galectin-9 is immunoprecitated from T cells by immobilized PD-L2. Lysates of total mouse T cell populations were mixed with immobilized IgG or PD-L2-Fc fusion protein. The bands were cut and digested for mass spectrophotometer analysis. Galectin-9 (2) was unique to immunoprecipitation with PD-L2.

FIG. 9: Western blot of Galectin-9 immunoprecitated from T cells, by immobilized PD-L2. Lysates of total mouse T cell populations were mixed with immobilized IgG or PD-L2-Fc fusion protein. The proteins on the gel were transferred to nitrocellulose which was immuno-labelled for galectin-9.

FIG. 10: sPD-L2 binds galectin-9 on T cells. Total T cell populations were isolated from spleens of naive mice and incubated with biotinylated sPD-L2 and APC-Streptavidin or PE-anti-galectin-9. T cells were also incubated with unlabelled anti-galectin-9 antibody, prior to labelling with biotinylated sPD-L2 and APC-Streptavidin. All samples were also labelled to identify CD4+ and CD8+ T cells.

FIG. 11: Soluble PD-L2 increases the differentiation of naive mouse CD4+ T cells and their T_{BET} levels mediated by Galectin 9. Naive CD4⁺ T cells were cultured with anti-CD3, IL-2 and stimuli shown on graph. (a) sPD-L2 increased the percentage of CD4+CD62L^{lo} cells which expressed T_{BET} and (b) the level of T_{BET} within cells compared to rat IgG treatment. This effect was blocked by anti-Galectin-9 (clone 108A) antibody which is determined to be a Galectin-9 inhibitor. Clone RG9.1 also increased the percentage of mouse CD4+CD62L^{lo} cells which expressed T_{BET}.

FIG. 12: Soluble synthetic PD-L2 and anti-galectin-9 antibody protect against symptoms of lethal malaria. Cohorts of B6 mice (n=3) were infected with lethal P. yoelii YM and on days 3, 5 and 7 mice were given either soluble PD-L2, anti-galectin-9 or human IgG (Control Ig). Mice were monitored daily and scored for symptoms of disease and survival. Mice were euthanized when score reached 4. The positive effect of sPDL2 on clinical score is mimicked and improved upon by RG1 which is determined to be a Galectin-9 stimulator (agonising antibody).

FIG. 13: Mouse PD-L2-Galectin-9 is highly stable and involves multimerisation of Galectin-9 and PD-L2. Octet red studies were undertaken to determine the biochemical nature of binding between Galectin-9 and PD-L2. The sPD-L2 was bound to the probe and it's interaction with sPD-1 and sGalectin-9 was measured. The PD-L2-PD1 binding curve shows that PD-L2 binds PD-1 in less than 0.02 sec (sensitivity of assay) and dissociates in less than 0.02 sec. The PD-L2-Gal-9 curve shows Galectin-9 binding takes 299.99 sec to associate and 614.21 sec to dissociate indicating a very stable interaction between PD-L2 and Galectin-9. The height of the peak shows a large aggregation of galectin-9 not seen with PD-1, indicating that Galectin-9 and PDL2 multimerise during binding.

FIG. 14: Cytokines secreted from mouse CD4⁺ T cells treated with mouse sPD-L2 or anti-galectin-9 antibody. CD4⁺ T cells were isolated from mouse spleens and cultured for 3 days with anti-CD3 and stimuli, supernatants collected to measure cytokines, Interferon-γ, IL-2 and TNF-α. Error bars represent SEM and data represents 1 of 2 experiments.

FIG. 15: (A) Cytokines secreted from human CD4⁺ T cells treated with human sPD-L2. CD4⁺ T cells were isolated from human PBMC and cultured for 3 days with PMA and ionomycin to mimic activation of the TCR. Cells were then cultured with sPD-L2 or control and, on day 3, supernatants collected to measure cytokines Interferon-γ (IFN-γ), IL-2, TNF-α and IL-4. Error bars represent SEM and data represents pooled data from 2 experiments. (B) IFN-γ secreted from human CD4⁺ T cells treated with anti-mouse galectin-9. CD4⁺ T cells were isolated from human PBMC and cultured for 3 days with a suboptimal concentration of anti-CD3 to mimic activation of the TCR. Cells were then cultured with human sPD-L2 or anti-mouse galectin-9 and, on day 3, supernatants collected to measure cytokines. Error bars represent SEM and data represents 1 experiment.

FIG. 16: Anti-galectin-9 activating antibodies, but not anti-Tim3 blocking antibodies, protect against lethal malaria. Mean percent parasitemia for a typical course of P. yoelii YM malaria in WT mice treated with Control rat IgG, blocking anti-Tim-3 antibody or activating anti-galectin-9 antibody on days 3, 5 and 7 post-infection. Error bars represent SEM and data represent 1 of 2 experiments for Tim3 and 1 of 3 experiments for anti-galectin-9.

FIG. 17: Anti-galectin-9 treatment reduces breast cancer progression. Cohorts of mice were ectopically transplanted with a (a) PYMT-derived or (b) EO771.LMB mammary carcinoma and treated with either control IgG or anti-galectin-9 antibody. Mice were monitored every 1-2 days to monitor tumour progression. QIMR-B ethics requires mice to be euthanized when tumours transplanted in the breast reached ˜525 mm². Error bars represent SEM.

FIG. 18: Blockade of PD-L2 inhibits the expansion of parasite-specific CD4⁺ T cells in the spleens of mice infected with P. yoelii 17XNL. Analysis of various parameters in WT mice infected with P. yoelii 17XNL and treated with Rat IgG or anti-PD-L2 blocking antibody. All data are shown in scatter plots with a bar representing the median value. (a, b) Numbers of Tbet-expressing CD4⁺CD62L^{hi} and CD4⁺CD62L^{lo} T cells per spleen on (a) day 7 (n=4) and (b) day 14 (n=7). (c) Numbers of CD4⁺ T cells that secreted interferon-γ (IFN-γ) in an ELISPOT culture in response to parasite antigen (MSP1₁₉) in the presence of naive DCs (n=7). (d, e) Levels of (d) IFN-γ and (e) IL-10 in the serum P. yoelii 17XNL-infected mice (n=7). (f) Numbers of CD4⁺ T cells expressing CD25 and FoxP3 (regulatory T cells) per spleen (n=7). The data for day 14 represents two pooled, independent experiments. Significance was analysed using the non-parametric Mann-Whitney U test based on 2-sided tail (*P<0.05; **P<0.005; ***P<0.0005). F tests found significantly different variances between groups.

FIG. 19: PD-L2 regulates Th1 immunity during P. yoelii 17XNL malaria. (A, B) Clinical symptom scores during typical course of P. yoelii 17XNL malaria in (A) WT mice and PD-L2KO mice (n=4) or (B) WT mice treated with Rat IgG or anti-PD-L2 blocking antibody (n=5). (C-F) Scatter plots show analysis of CD4 T⁺ cells in WT mice treated with Rat IgG or anti-PD-L2 blocking antibody or PD-L2KO mice infected with P. yoelii 17XNL for 14 days. (C) Mean numbers of T_{bet}-expressing CD4⁺CD62L^{hi} or CD4⁺CD62L^{lo} T cells per spleen. (D) Mean numbers of CD4⁺ T cells per spleen, that secreted IFN-γ in an ELISPOT culture in response to parasite antigen (MSP1₁₉), in the presence of naive DCs. (E-F) Mean numbers of CD8⁺ T cells per spleen that secreted IFN-γ in an ELISPOT culture in response to parasite antigen (Pb1), in the presence of naive DCs. (E) Cells taken on day 14 p.i. with P. yoelii 17XNL, with and without PD-L2 blockade (n=7). (F) Cells taken on day 14 p.i. with P. yoelii 17XNL, from PD-L2KO mice and controls. The data are pooled from 2 independent experiments except for PD-L2KO mice which was done once. Error bars represent SEM (*P<0.05). Significance was analysed using the non-parametric Mann-Whitney U test.

FIG. 20: sPD-L2 mediates protection and survival through CD4⁺ T cells (a) Survival curves and (b-d) Mean percent parasitemia in WT mice treated with control human IgG (hIg) or sPD-L2 on days 3, 5 and 7 post-infection with P. yoelii YM. Mice were then co-treated with (b) rat Ig, (c) depleting anti-CD4 antibody or (d) depleting anti-CD8 antibody (n=4) beginning on day 1 p.i. and every 3-4 days until day 14-18 p.i. The data represent one of two independent experiments that obtained similar results. Significance of survival between sPD-L2⁺ rat IgG treated group and the control group (given rat and human IgG) or sPD-L2 treated group with CD4⁺ T cell depletion was analysed using Log-rank (Mantel-Cox) test based on data from pooled experiments.

FIG. 21: sPD-L2 protects mice from lethal malaria by promoting Th1 CD4⁺ and CD8+ T cell functions. Analysis of various parameters (day 7 p.i.) in WT mice infected with P. yoelii YM and treated with control human IgG or sPD-L2 on days 3, 5 and 7 p.i. (n=8). All data are shown in scatter plots with a bar representing the median value. (a) Numbers of CD4⁺ T cells that secreted IFN-γ in ELISPOT cultures in response to parasite antigen MSP1₁₉ in the presence of naive DCs; (b) Numbers of CD4⁺ T cells that proliferated in cultures in response to parasite antigen MSP1₁₉ in the presence of naive DCs, measured by incorporation of EdU; (c) Numbers of CD4⁺ T cells expressing CD25 and FoxP3 per spleen. (d) Numbers of parasite-specific Pb1-tetramer⁺ CD8⁺ T cells per spleen, and (e) Numbers of CD8⁺ T cells that secreted IFN-γ in cultures in response to parasite peptide Pb1 in the presence of naive DCs (as determined by ELISPOT); (f) Numbers of CD8⁺ T cells which expressed CD11a a marker of recent activation and granzyme B. The data represent two pooled independent experiments, except for tetramer and granzyme B labelling which was undertaken once. Significance was analysed using the non-parametric Mann-Whitney U test based on 2-sided tail (*P<0.05; **P<0.005). F tests found significantly different variances between groups.

### DETAILED DESCRIPTION

PD-L2 is a ligand for programmed death receptor-1 (PD1) and RGMb and it is proposed herein that Galectin-9 (Gal-9) is a hitherto unknown receptor for PD-L2. Soluble PD-L2 treated mice do not die when challenged with a lethal malaria strain and it is proposed that PD-L2 mediates protection via CD4+ T cells, as PD-L2-mediated protection is lost when CD4+ T cells are depleted. Accordingly, it is proposed that administering soluble PD-L2 (sPDL2) or an agonising anti-Galectin-9 antibody can act as an immunostimulant and/or initiate a Th1-mediated immune response and/or immunological memory. This may have efficacy in stimulating immune responses to cancer, infectious agents and parasites, including the generation and maintenance of immunological memory, particularly against cancers. It is also proposed that administering blocking or antagonist antibodies to Galectin-9 may prevent or inhibit the effects seen with PD-L2. Antibodies that bind to PD-L2 and block its interaction with Galectin-9 may have a similar effect to Galectin-9 antagonist antibodies. This may assist in suppressing immunity such as may be useful in treating or preventing autoimmune disease, inflammation and/or allergy.

For the purposes of this invention, by “isolated” is meant material that has been removed from its natural state or otherwise been subjected to human manipulation. Isolated material may be substantially or essentially free from components that normally accompany it in its natural state, or may be manipulated so as to be in an artificial state together with components that normally accompany it in its natural state. Isolated material may be in native, chemical synthetic or recombinant form.

By “protein” is meant an amino acid polymer. The amino acids may be natural or non-natural amino acids, D- or L-amino acids as are well understood in the art.

A “peptide” is a protein having no more than fifty (50) amino acids.

A “polypeptide” is a protein having more than fifty (50) amino acids.

As used herein “Galectin-9” or “Gal-9” refers to a protein of the galectin family of proteins defined by their binding specificity for β-galactoside sugars, such as N-acetyllactosamine (Galβ1-3GlcNAc or Galβ1-4GlcNAc). These proteins are also termed 5-type lectins due to their dependency on disulphide bonds for stability and carbohydrate binding. There have been 15 galectins discovered in mammals, encoded by the LGALS genes, of which Galectin-1, -2, -3, -4, -7, -8, -9, -10, -12 and -13 have been identified in humans. Human Galectin 9 typically comprises a 355 amino acid sequence (referred to as the canonical or “long form” sequence), although there is a “short form” variant lacking residues 149-180. Suitably, in the context of the invention Galectin-9 is expressed by a lymphocyte or an NK cell. The lymphocyte may be CD4+ T cell, a CD8+ T cell or a B cell. A non-limiting example of a human Galectin-9 amino acid sequence may be found under Uniprot KB accession number O00182 and a non-limiting example of a mouse Galectin-9 amino acid sequence may be found under Uniprot KB accession number O08573.

As used herein an “antibody” is or comprises an immunoglobulin. The term “immunoglobulin” includes any antigen-binding protein product of a mammalian immunoglobulin gene complex, including immunoglobulin isotypes IgA, IgD, IgM, IgG and IgE and antigen-binding fragments thereof. Included in the term “immunoglobulin” are immunoglobulins that are chimeric or humanised or otherwise comprise altered or variant amino acid residues, sequences and/or glycosylation, whether naturally occurring or produced by human intervention (e.g. by recombinant DNA technology).

Antibody fragments include Fab and Fab′2 fragments, diabodies, triabodies and single chain antibody fragments (e.g. scVs), although without limitation thereto. Typically, an antibody comprises respective light chain and heavy chain variable regions that each comprise CDR 1, 2 and 3 amino acid sequences. A preferred antibody fragment comprises at least one light chain variable region CDR and/or at least one heavy chain variable region CDR.

Antibodies and antibody fragments may be polyclonal or preferably monoclonal. Monoclonal antibodies may be produced using the standard method as for example, described in an article by Köhler & Milstein, 1975, Nature 256, 495-497, or by more recent modifications thereof as for example described in Chapter 2 of Coligan et al., CURRENT PROTOCOLS IN IMMUNOLOGY, by immortalizing spleen or other antibody producing cells derived from a production species which has been inoculated with Galectin-9 or a fragment thereof. It will also be appreciated that antibodies may be produced as recombinant synthetic antibodies or antibody fragments by expressing a nucleic acid encoding the antibody or antibody fragment in an appropriate host cell. Recombinant synthetic antibody or antibody fragment heavy and light chains may be co-expressed from different expression vectors in the same host cell or expressed as a single chain antibody in a host cell. Non-limiting examples of recombinant antibody expression and selection techniques are provided in Chapter 17 of Coligan et al., CURRENT PROTOCOLS IN IMMUNOLOGY and Zuberbuhler et al., 2009, Protein Engineering, Design & Selection 22 169.

Antibodies and antibody fragments may be modified so as to be administrable to one species having been produced in, or originating from, another species without eliciting a deleterious immune response to the “foreign” antibody. In the context of humans, this is “humanization” of the antibody produced in, or originating from, another species. Such methods are well known in the art and generally involve recombinant “grafting” of non-human antibody complementarity determining regions (CDRs) onto a human antibody scaffold or backbone.

In some embodiments, the antibody or antibody fragment is labelled.

The label may be selected from a group including a chromogen, a catalyst, biotin, digoxigenin, an enzyme, a fluorophore, a chemiluminescent molecule, a radioisotope, a drug or other chemotherapeutic agent, a magnetic bead and/or a direct visual label.

An aspect of the invention provides a method of modulating immunity in a mammal including the step of modulating Galectin-9 activity in the mammal to thereby modulate immunity in the mammal.

In one embodiment, “modulating immunity” means promoting or enhancing immunity in the mammal. In this context, Galectin-9 activity is stimulated or increased, such as by an agonist.

In another embodiment, “modulating immunity” means at least partly suppressing, inhibiting or preventing immunity in the mammal. In this context, Galectin-9 activity is at least partly blocked or inhibited, such as by a Galectin-9 antagonist or inhibitor.

One particular aspect of the invention therefore provides a method of promoting or enhancing immunity in a mammal including the step of activating, increasing or stimulating Galectin-9 activity in the mammal to thereby stimulate or enhance immunity in the mammal.

Suitably, the method includes the step of administering a Galectin-9 agonist to the mammal to thereby activate or stimulate Galectin-9 activity in the mammal.

In this context by “agonist” is meant a molecule that at least partly activates, increases or stimulates a Galectin-9 activity. The agonist may be a natural ligand for Galectin-9 such as PD-L2 or may mimic the action of a natural ligand such as PD-L2. In one particular embodiment, the method includes the step of administering soluble PD-L2 or a biologically active fragment thereof to the mammal to thereby activate or stimulate Galectin-9 activity in the mammal. Suitably, PD-L2 is multimeric, preferably comprising n monomers, wherein n≥3. Preferably, multimeric PD-L2 comprises three, four, five, six, seven or eight PD-L2 monomers. In one particular embodiment, PD-L2 comprises eight monomers (i.e. n=8 or octomeric). In this context, multimeric PD-L2 may be induced or formed covalently such as by chemical-crosslinking of monomers including use of linker amino acids or peptides to facilitate covalent coupling of each monomer. In other embodiments, the effect of multimeric PD-L2 may be mimicked by an agent such as a peptide or nucleic acid (e.g. an oligonucleotide) aptamer or by a bi-specific antibody which binds both PD-L2 and Galectin-9 or mimicked by an agent such as a peptide or nucleic acid (e.g. an oligonucleotide) aptamer. The agonist may be any other molecule that can bind to Galectin-9 to thereby activate or stimulate Galectin-9 activity, such as an agonist antibody or antibody fragment. In one particular embodiment, the method includes the step of administering to the mammal an agonist antibody or antibody fragment that binds Galectin-9 to thereby activate or stimulate Galectin-9 activity in the mammal.

In some embodiments, the agonist may stimulate or enhance an immune response upon administration to a mammal. The immune response may include the induction of immunological memory against cancer or a pathogen such as one that causes infectious and/or parasitic, particularly where the pathogen evades the immune system by avoiding, erasing or evading immunological memory. A non-limiting example is malaria which erases immunological memory to thereby allow later re-infection.

In the context of cancer, administration of the agonist to cancer patients may create, induce and/or maintain immunological memory so that tumour cells are recognized as foreign when non-self signals are otherwise low or lacking.

In another embodiment, the Galectin-9 agonist may be administered as an adjuvant in combination with an immunogen in a vaccine or other immunogenic composition. This may boost the effectiveness of the vaccine or immunogenic composition and might also remove or minimize the need for booster vaccination. In a particular form of this embodiment, administration of the agonist may rescue or revive a failed or sub-optimal vaccine or vaccination which does not sufficiently stimulate an immunological memory of the immunogen or pathogen. The immunogen may be a component molecule of a pathogen (e.g. a cell surface protein, immunogenic peptide or other component thereof such as in a “subunit vaccine”, a polytope comprising a plurality of B- and/or T-epitopes, VLPs, capsids, or capsomeres), an inactivated pathogen (e.g. an inactivated virus, attenuated parasite-infected RBC, or attenuated bacterium) or any other molecule or structure capable of eliciting an immune response to the pathogen. For example, reference is made to the Examples demonstrating the efficacy of administering PD-L2 and anti-Galectin-9 antibody agonist in improving malaria immunization.

Administration of the agonist to a mammal may stimulate naive T cells to make a Th1 lineage choice and/or commitment. As will be understood by persons skilled in the art, the Th1 lineage includes CD4+ T cells that produce and secrete one of more factors including interferon γ (IFN-γ), IL-2 and TNF-α, although without limitation thereto. Th1 cells are particularly important in the immune response against intracellular bacteria, protozoan parasites and viruses. Th1 cells are triggered by IL-12 and IL-2 and in turn stimulate immune effector cells such as macrophages, granulocytes, CD8+ T cells, IgG-expressing B cells, dendritic cells and other CD4+ T cells.

From the foregoing, it will be appreciated that activation or stimulation of Galectin-9, such as by an agonist disclosed herein, may treat or prevent a disease disorder or condition in a mammal.

As used herein, “treating”, “treat” or “treatment” refers to a therapeutic intervention, course of action or protocol that at least ameliorates a symptom of the disease, disorder or condition after symptoms have at least started to develop. As used herein, “preventing, “prevent” or “prevention” refers to therapeutic intervention, course of action or protocol initiated prior to the onset of a symptom of the disease, disorder or condition so as to prevent, inhibit or delay or development or progression of the disease, disorder or condition or the symptom. Such preventative therapies may be referred to as “prophylaxis” or “prophylactic” treatments. In a specific embodiment, immunization or vaccination is a preventative or prophylactic immunotherapy.

In a broad embodiment, the disease, disorder or condition is caused by a pathogen. The pathogen may be a virus, bacterium or parasite. A non-limiting example of a parasite includes protozoa such as malaria parasites inclusive of Plasmodium spp such as P. falciparum, P. ovale, P. knowlesii, P. malariae and P. vivax, although without limitation thereto. Other parasites include Babesia spp, Entamoeba spp, Giardia spp and Trypanosomes inclusive of Leishmania spp, although without limitation thereto.

Non-limiting examples of viral pathogens include human immunodeficiency virus (HIV), Ebola virus, influenza virus, herpes virus, papilloma virus, measles virus, mumps virus, hepatitis B virus, rubella virus, rhinovirus, flaviviruses such as hepatitis C virus (HCV), West Nile virus, Japanese encephalitis virus and Dengue virus, cytomegalovirus (CMV) and Epstein Barr Virus (EBV), although without limitation thereto.

Non-limiting examples of bacterial pathogens may be of genera such as Neisseria, Bordatella, Pseudomonas, Corynebacterium, Salmonella, Streptococcus, Shigella, Mycobacterium, Mycoplasma, Clostridium, Helicobacter, Borrelia, Yersinia, Legionella, Hemophilus, Rickettsia, Listeria, Brucella, Vibrio and Treponema, including species such as Staphylococcus aureus, Staphylococcus epidermidis, Helicobacter pylori, Bacillus anthracia, Bordatella pertussis, Corynebacterium diptheriae, Corynebacterium pseudotuberculosis, Clostridium tetani, Clostridium botulinum, Streptococcus pneumoniae, Streptococcus mutans, Streptococcus oralis, Streptococcus parasanguis, Streptococcus pyogenes, Streptococcus Listeria monocytogenes, Hemophilus influenzae, Pasteurella multicida, Shigella dysenteriae, Mycobacterium tuberculosis, Mycobacterium leprae, Mycobacterium asiaticum, Mycobacterium intracellulare, Mycoplasma pneumoniae, Mycoplasma hominis, Neisseria meningitidis, Neisseria gonorrhoeae, Rickettsia rickettsii, Brucella abortis, Brucella canis, Brucella suis, Legionella pneuophila, Klebsiella pneumoniae, Pseudomonas aeruginosa, Treponema pallidum, Treponema pertanue, Chlamydia trachomatis, Vibrio cholerae, Treponema carateum, Salmonella typhimurium, Salmonella typhi, Borrelia burgdorferi, and Yersinia pestis, although without limitation thereto.

In another broad embodiment, the disease, disorder or condition is cancer. As generally used herein, the terms “cancer”, “tumour”, “malignant” and “malignancy” refer to diseases or conditions, or to cells or tissues associated with the diseases or conditions, characterized by aberrant or abnormal cell proliferation, differentiation and/or migration often accompanied by an aberrant or abnormal molecular phenotype that includes one or more genetic mutations or other genetic changes associated with oncogenesis, expression of tumour markers, loss of tumour suppressor expression or activity and/or aberrant or abnormal cell surface marker expression. Non-limiting examples of cancers and tumours include sarcomas, carcinomas, adenomas, leukaemias and lymphomas, lung cancer, colon cancer, liver cancer, oesophageal cancer, stomach cancer, pancreatic cancer, neuroblastomas, glioblastomas and other neural cancers, brain, breast cancer, cervical cancer, uterine cancer, head and neck cancers, kidney cancer, prostate cancer and melanoma. Suitably, the cancer is responsive to activation or stimulation of Galectin-9, such as by an agonist disclosed herein. In some embodiments, the cancer is responsive to induction or enhancement of immunological memory resulting from activation or stimulation of Galectin-9.

Another particular aspect of the invention provides a method of at least partly suppressing or preventing immunity in a mammal including the step of at least partly inhibiting or blocking Galectin-9 activity in the mammal to thereby suppress or prevent immunity in the mammal.

Suitably, the method includes the step of administering a Galectin-9 inhibitor or antagonist to the mammal to thereby inhibit or block Galectin-9 activity in the mammal. Preferably, the inhibitor or antagonist at least partly prevents or interferes with a binding interaction between PD-L2 and Galectin-9. Additionally or alternatively, the Galectin-9 inhibitor or antagonist at least partly prevents or interferes with Galectin-9 signalling that would normally occur in response to PD-L2 binding. In some embodiments, the Galectin-9 inhibitor or antagonist may be an agent that directly binds Galectin-9 (such as an anti-Galectin-9 antibody or antibody fragment) or may be an agent that directly binds PD-L2 (such as an anti-PD-L2 antibody fragment) to inhibit or block PD-L2 multimerization and/or binding to Galectin-9. In particular embodiments, the Galectin-9 inhibitor or antagonist may include: (i) soluble Galectin-9 or an inhibitory fragment thereof; (ii) an antagonist antibody or antibody fragment or other agent that binds Galectin-9 to thereby inhibit or block binding between PD-L2 and Galectin-9 and/or Galectin-9 signalling; (iii) monomeric or dimeric PD-L2 that inhibits or blocks binding between PD-L2 and Galectin-9 and/or Galectin-9 signalling; (iv) an antibody or antibody fragment or other agent that binds to PD-L2 and thereby prevents PD-L2 from binding to Galectin-9; and/or (v) an antibody or antibody fragment or other agent that binds to PD-L2 to prevent or inhibit PD-L2 multimerization.

In one embodiment, the method therefore includes the step of administering soluble Galectin-9 or an inhibitory fragment thereof to the mammal to thereby inhibit or block binding between PD-L2 and Galectin-9 in the mammal. In one embodiment, the method includes the step of administering to the mammal an antagonist antibody or antibody fragment that binds Galectin-9 to thereby inhibit or block binding between PD-L2 and Galectin-9 and/or Galectin-9 signalling, in the mammal. In another embodiment, the method includes the step of administering to the mammal monomeric or dimeric PD-L2 to thereby inhibit or block binding between PD-L2 and Galectin-9 and/or Galectin-9 signalling, in the mammal. In a further embodiment the method includes the step of administering to the mammal an antibody or antibody fragment that binds to PD-L2 and thereby prevents PD-L2 from binding to Galectin-9. In a still further embodiment, the method includes the step of administering to the mammal an antibody or antibody fragment or other agent that binds to PD-L2 to prevent or inhibit PD-L2 multimerization in the mammal.

In certain embodiments, suppressing or preventing immunity in the mammal may facilitate or assist prevention or treatment of a disease, disorder or condition. In particular embodiments the disease, disorder or condition may be an autoimmune disease, disorder or condition, an inflammatory disease, disorder or condition inclusive of an allergic disease, disorder or condition.

It will be appreciated that there may be overlap between autoimmune and inflammatory diseases, disorders and conditions due to commonality in the underlying immunological mechanisms that lead to autoimmune and/or inflammatory diseases, disorders and conditions. However, by way of example only, autoimmune diseases, disorders or conditions include Sjogren's syndrome, type I diabetes, ankylosing spondylitis, Hashimoto's thyroiditis, Chrohn's disease, amyotrophic lateral sclerosis, systemic lupus erythematosus, myasthenia gravis, multiple sclerosis, Graves disease, Addison's disease, Behçet's syndrome, VogtKoyanagi-Harada (VKH) disease, rheumatoid arthritis and psoriatic arthritis, although without limitation thereto. Non-limiting examples of inflammatory diseases, disorders or conditions include inflammatory bowel disease, atherosclerosis, pelvic inflammatory disease, celiac disease, asthma, chronic obstructive pulmonary disease and allergies, although without limitation thereto.

In one particular embodiment, the disease, disorder or condition is responsive to blocking or inhibition of T-bet or a signalling pathway comprising T-bet.

Although not wishing to be bound by any particular theory, the T-box transcription factor T-bet is a key regulator of type 1-like immunity, playing critical roles in the establishment and/or maintenance of effector cell fates in T and B lymphocytes, as well as dendritic cells and natural killer cells. T-bet may play a role in the maintenance of Th1 effector function and differentiation, including IFN-γ production in CD4 and γδ T cells, although without limitation thereto. For example, a T-bet deficiency protects against, while T-bet overexpression promotes, autoimmune and/or inflammatory diseases. As will be described in more detail in the Examples, blocking the PD-L2/galectin-9 pathway blocks T-bet, which therefore has the potential to provide a new method for treatment of autoimmune and/or inflammatory diseases.

Administration of Galectin-9 agonists, antagonists and inhibitors as hereinbefore described may be practiced by administering a pharmaceutical composition comprising Galectin-9 agonists, antagonists and inhibitor together with a suitable carrier, diluent or excipient.

In general terms, a carrier, diluent or excipient may be a solid or liquid filler, diluent, buffer, binder or encapsulating substance that may be safely used in systemic administration. Depending upon the particular route of administration, a variety of carriers, diluents and excipients well known in the art may be used. These may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts such as mineral acid salts including hydrochlorides, bromides and sulfates, sugars, sugar alcohols, organic acids such as acetates, propionates and malonates, and pyrogen-free water. A useful reference describing pharmaceutically acceptable carriers, diluents and excipients is Remington's Pharmaceutical Sciences (Mack Publishing Co. NJ USA, 1991).

In some embodiments, the composition may further comprise one or more immunomodulatory agents inclusive of adjuvants and immunostimulatory nucleic acids including but not limited to TLR agonists, lipopolysaccharide and derivatives thereof such as MPL, Freund's complete or incomplete adjuvant, hexadecylamine, octadecylamine, octadecyl amino acid esters, lysolecithin, dimethyldioctadecylammonium bromide, N,N-dicoctadecyl-N′, N′bis(2-hydroxyethyl-propanediamine), methoxyhexadecylglycerol, and pluronic polyols; polyamines such as pyran, dextransulfate, poly IC carbopol, peptides such as muramyl dipeptide and derivatives, dimethylglycine, tuftsin, oil emulsions, mineral gels such as aluminum phosphate, aluminum hydroxide or alum, lymphokines, Imiquimod, Guardiquimod, QuilA and immune stimulating complexes (ISCOMS).

Any safe route of administration may be employed for providing a subject with compositions comprising the Galectin-9 agonist, antagonist or inhibitor. For example, oral, rectal, parenteral, sublingual, buccal, intravenous, intra-articular, intra-muscular, intra-dermal, subcutaneous, inhalational, intraocular, intraperitoneal, intracerebroventricular, transdermal, and the like may be employed.

The concentration or amount of the Galectin-9 agonist, antagonist or inhibitor to be administered to a mammal may be readily determined by persons skilled in the art and will take into account factors such as the nature of the disease, disorder or condition to be treated and/or the body weight, age, sex and/or the general health and well-being of the mammal.

In one embodiment, the pharmaceutical composition may be a vaccine or other immunogenic composition. Suitably, the vaccine or immunogenic composition comprises a Galectin-9 agonist, a suitable carrier, diluent or excipient and an immunogen that is capable of eliciting an immune response in a mammal. Preferably, the immune response is a protective immune response that includes the elicitation of immunological memory. The immunogen may be a component molecule of a pathogen (e.g. a cell surface protein, immunogenic peptide or other component thereof such as in a “subunit vaccine”, a polytope comprising multiple B- and/or T-epitopes, VLPs, capsids, or capsomeres), an inactivated pathogen (e.g. an inactivated virus, attenuated parasite-infected RBC, or attenuated bacterium) or any other molecule capable of eliciting an immune response to the pathogen. For example, reference is made to the Examples demonstrating the efficacy of administering PD-L2 and anti-Galectin-9 antibody agonist in improving malaria immunization.

A further aspect of the invention provides a method of designing, screening, engineering or otherwise producing a Galectin-9 agonist, inhibitor and/or antagonist, said method including the step of (i) determining whether a candidate molecule is an agonist which activates or stimulates Galectin-9 activity and is thereby capable of stimulating or enhancing immunity in a mammal; or (ii) determining whether a candidate molecule is an antagonist or inhibitor which blocks or inhibits Galectin-9 activity and is thereby capable of at least partly suppressing or preventing immunity in a mammal.

Broadly, Galectin-9 agonists, inhibitors and/or antagonists designed, screened, engineered or otherwise produced according to this method may be capable of stimulating or enhancing immunity or in a mammal or be capable of at least partly suppressing or preventing immunity in a mammal as hereinbefore described.

In one particular embodiment, in step (i) the candidate molecule mimics PD-L2 stimulation or activation of Galectin-9.

In one particular embodiment, in step (ii) the candidate molecule at least partly blocks or inhibits PD-L2 stimulation or activation of Galectin-9.

The candidate molecule may be a protein, inclusive of peptides or polypeptides such as an antibody or antibody fragment as hereinbefore described, a small organic molecule, a carbohydrate such as a mono-, di-, tri- or poly-saccharide, a lipid, a nucleic acid, an aptamer or any molecule which comprises one or more of these, although without limitation thereto.

Non-limiting examples of techniques applicable to the design and/or screening of candidate modulators may employ X-ray crystallography, NMR spectroscopy, computer assisted screening of structural databases, computer-assisted modelling or biochemical or biophysical techniques which detect molecular binding interactions, as are well known in the art.

Biophysical and biochemical techniques which identify molecular interactions include competitive radioligand binding assays, co-immunoprecipitation, fluorescence-based assays including fluorescence resonance energy transfer (FRET) binding assays, electrophysiology, analytical ultracentrifugation, label transfer, chemical cross-linking, mass spectroscopy, microcalorimetry, surface plasmon resonance and optical biosensor-based methods and quantum dot biosensors such as provided in Chapter 20 of CURRENT PROTOCOLS IN PROTEIN SCIENCE Eds. Coligan et al., (John Wiley & Sons, 1997-2013) Biochemical techniques such as two-hybrid and phage display screening methods are provided in Chapter 19 of CURRENT PROTOCOLS IN PROTEIN SCIENCE Eds. Coligan et al., (John Wiley & Sons, 1997-2013).

Accordingly, an initial step of the method may include identifying a plurality of candidate molecules that are selected according to broad structural and/or functional attributes, such as an ability to bind Galectin-9 and/or compete with or otherwise PD-L2 binding to Galectin-9 or prevent or inhibit PD-L2 multimerization.

The method may include a further step that measures or detects a change in one or more biological activities associated with Galectin-9 in response to the candidate molecule(s). These may include activation or inhibition of Galectin-9 intracellular signalling, cytokine production, protection from tumour challenge, enhancement of immunization with a pathogen or pathogen-derived molecule (e.g. a vaccine), suppression of autoimmune, inflammatory or allergic responses, induction of T cell memory in vitro or in vivo, although without limitation thereto. Methods and protocols for measuring or detecting such changes in one or more biological activities associated with Galectin-9 are well known to persons skilled in the art, at least some of which are provided in detail in the Examples to follow.

It will be appreciated that Galectin-9 agonists, antagonists and/or inhibitors may be useful according to the methods hereinbefore described.

The invention disclosed herein may be practiced in any mammal that expresses Galectin-9 or a functional homologue thereof. Preferably, the mammal is a human.

So that particular embodiments of the invention may be readily understood and put into practical effect, reference is made to the following non-limiting examples.

### Examples

### PD-L2 and Galectin-9

Scientific consensus has been that sPD-L2 may have beneficial effects but this is via ligand competition for PD1: When PD-L1 binds PD1 it shuts down the immune response while PDL2 may have an opposing effect by competing with PD-L1 for PD1 binding. There appear to be few reports of positive stimulatory effect of PD-L2 per se. Regarding, Galectin-9, this is considered to be a ligand for Tim3, wherein Tim3 is a immunomodulator that contributes to T cell exhaustion which is induced or mediated by Galectin-9 binding. To avoid T cell exhaustion, much development work is geared towards blocking the Galectin-9/Tim3 interaction with antibodies, thereby boosting the immune response. This runs somewhat counter to the present invention which seeks to agonise Galectin-9 to achieve an improved immune response. However, a recent paper found that Galectin-9 and Tim3 are not interacting (at least in humans) so the consensus may be changing (Leitner et al., 2013). In the review by Gabriel et al., 2009 it was suggested that administration of Galectin-9 to mice, rabbits and rats has the opposite effect to that described herein (at least in activated T cells) and is expected to also have the opposite effect in naïve T cells. Furthermore, Gabriel et al. concluded that in the thymic microenvironment, galectin 1, galectin 3, galectin 8 and galectin 9 induce apoptosis in double-negative (CD4⁻CD8⁻) or double-positive (CD4⁺CD8⁺) thymocytes, suggesting a possible role for these galectins in regulating central tolerance. Again, this view is in contrast to the present invention.

### Malaria

Several diseases such as malaria, HIV and TB cause morbidity and mortality in millions of individuals around the globe, each year. The development of a vaccine has proven to be greatly challenging as these pathogens have evolved several mechanisms to evade immunity. The Programmed cell death-1 (PD-1) pathway has been implicated as a mechanism by which the HIV and Plasmodium spp (the causative agent for malaria) escape immunity. We thus used mouse models of malaria to investigate how this pathway could compromise immunity.

Malaria infects 300-500 million individuals each year and kills millions. There have been >40 clinical trials for malaria vaccines, mostly based on antibody-mediated protection, but only one reached Phase Mb. Even life-long exposure to malaria may not induce protective antibody responses (Egan et al., 1995; Egan et al., 1996) and young children infected with blood-stage Plasmodium falciparum (Pf) experience rapid declines in pre-existing anti-malarial antibody levels (Akpogheneta et al., 2008; Kinyanjui et al., 2007). More detailed studies using a protein microarray containing approximately 23% of P. falciparum protein proteome probed with plasma from 220 individuals confirmed that antibody reactivity to these proteins rose dramatically during the malaria season but was short-lived (Crompton et al., 2010). A previous study which measured antigen-specific memory B cells (MBC) from children in malaria-endemic areas found that multiple exposures to malaria did not generate stable populations of circulating antigen-specific MBC (Dorfman et al., 2005). Further, longitudinal studies recently showed that both Pf-specific MBC and antibody titres increased after acute malaria, then contracted to a point slightly higher than pre-infection levels within 6 months indicating an inefficient, stepwise expansion of both the Pf-specific MBC and long-lived antibody compartments which may explain why immunity is poor in children and takes several years to develop (Weiss et al., 2010). In contrast to these studies based predominantly in Africa, in Thailand, where the endemicity of malaria is far lower, individuals who were known to have had a documented clinical attack of P. falciparum and/or P. vivax in the past 6 years had antigen-specific antibodies and/or stable frequencies of antigen-specific MBCs (Wipasa et al., 2010).

CD4⁺ T cells consist of several helper-subtypes which shape immune responses against particular pathogens. During malaria, CD4⁺ T cell subsets have multiple roles in protection, pathogenesis and also escape from immune responses. CD4⁺ T cells have been demonstrated to be the major source of both Interferon-γ (IFN-γ) and tumour necrosis factor alpha (TNF-α) during experimental malaria in mice (Muxel et al., 2011) which are implicated in protection against this disease. Studies in mice infected with P. chabaudi malaria have shown that IFN-γ and TNF-α cooperatively induce nitric oxide synthase expression in the spleen to control peak parasite burden (Jacobs et al., 1996). Similarly, in humans, early IFN-γ responses to Pf correlate with better anti-parasite immunity (McCall et al., 2010). IFN-γ contributes to a vast network of protective responses against malaria, summarised by (McCall and Sauerwein, 2010). Of particular note is a study which investigated the effects of chronic malaria on MSP1-specific transgenic CD4⁺ T cells (Stephens and Langhorne, 2010). These parasite-specific T cells were seeded into Thy1.1 congenic mice which were then infected with 10⁵ Plasmodium chabaudi infected red cells. One half of the mice were treated with Chloroquine on days 30-34 to clear chronic malaria. After 60 days, flow cytometric analysis of transgenic T cells found that approximately 25% of memory CD44⁺IL-7R⁺ CD4⁺ T cells were lost in untreated mice compared to drug-treated mice which had cleared the infection (Stephens and Langhorne, 2010). This study highlights that on-going infections cause a loss of some parasite-specific memory T cells capable of protection from re-infection.

### Programmed Death-1 (PD-1) and Malaria

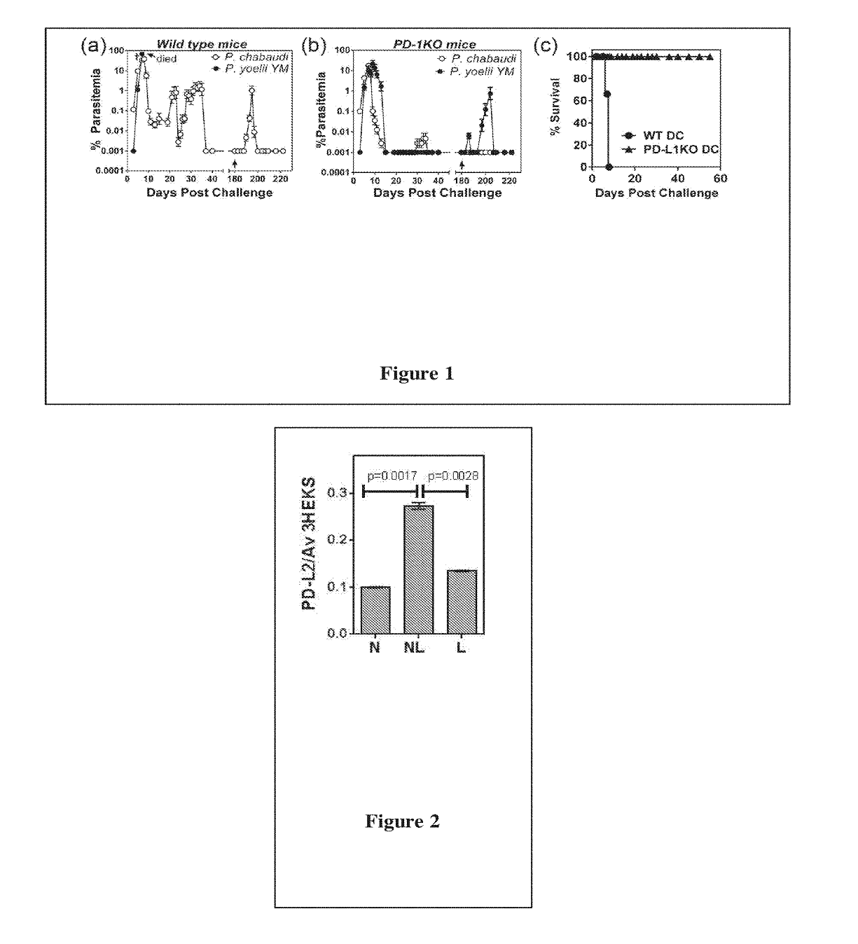

PD-1 has been implicated in the pathogenesis of malaria. To understand the role of PD-1 in immunity against chronic and lethal malaria, and long term protection from re-infection, cohorts of C57/Bl6 (WT) mice and C57/Bl6 mice with PD-1 gene deleted (PD-1KO) were infected with non-lethal 10⁵ P. chabaudi (chronic malaria) or lethal P. yoelii YM parasitized red cells (pRBC) and blood was examined for parasitemia every 1-2 days. After 40 days, all surviving mice were rested for 140 days to allow primary immune cells to subside with only memory cells surviving. These mice were then re-infected with the corresponding parasite at day 180 (arrows in FIGS. 1a and b). We found that all WT mice infected with non-lethal P. chabaudi cleared the primary infection in approximately 35 days (FIG. 1a). When these WT mice were re-infected at day 180, all mice developed parasitemia although at a much lower level than the first infection (FIG. 1a). In contrast, PD-1KO mice cleared P. chabaudi infections in 15 days with only 20% of mice experiencing low grade recrudescent infections around day 30 (FIG. 1b). On re-infection, 9/9 PD-1-KO mice showed no parasitemia (FIG. 1b) and had sterile immunity when blood was transferred to naive mice (data not shown).

When WT mice were infected with lethal P. yoelii YM, all mice died within 7 days of infection (FIG. 1a). In contrast, 10/10 PD-1KO mice survived lethal P. yoelii YM infections and re-infections after 180 days. Significantly, only 40% of re-challenged mice experienced low level parasitemia (FIG. 1b). These studies show that the PD-1 pathway drives chronic and lethal malarias and prevents optimal long-term protection against re-infection.

### Exhaustion of CD4⁺ T Cells During Malaria

One of the first studies to examine PD-1 expression during malaria used a mouse model to show PD-1 expression on IL-7R^{lo}-expressing CD4⁺ and CD8⁺ T cells (Chandele et al., 2011). These PD-1-expressing cells (especially CD8⁺ T cells) were almost completely lost within 30 days of infection (Chandele et al., 2011). The study did not however measure functional responses to identify T cell exhaustion. Similarly, subsequent studies showed that PD-1 was also expressed on CD4⁺ (Butler et al., 2012; Illingworth et al., 2013) and CD8⁺ T cells (Illingworth et al., 2013) in blood of Pf-infected individuals in Mali and Kenya, but no functional evidence of exhaustion was provided.

To validate these observations, a murine model of blood stage malaria was adopted to explore the effects of increased expression of PD-1 and LAG-3 on CD4⁺ T cells (Butler et al., 2012). The combined blockade of PD-L1 and LAG-3 inhibitory molecules with antibodies, during P. yoelii and P. chabaudi malaria in mice accelerated clearance of parasitemia (Butler et al., 2012). This dual blockade of PD-L1 and Lag-3 improved CD4⁺ Follicular T helper cell (T_{FH}) numbers which correlated with enhanced antibody-mediated immunity (Butler et al., 2012). Moreover, infected mice treated with the anti-malarial drug chloroquine at day 8 and 9 post infection, showed a lower level of CD4⁺ T cell dysfunction (Butler et al., 2012). These studies showed that lymphocyte exhaustion modulated immunity against malaria.

Subsequent studies used mice with a deletion of PD-1 (PD-1KO) to conclusively determine if PD-1 had a role in modulating immunity, given that PD-L1 can interact specifically with both B7-1 (Butte et al., 2007) and PD-1 (Iwai et al., 2003) to inhibit T cell activation. P. chabaudi malaria was investigated as this infection develops into chronic infections. It was shown that PD-1 mediated a reduction in the capacity of parasite-specific CD4⁺ T cells to proliferate and secrete IFN-γ and TNF-α during the chronic phase of malaria (Day 35) indicating exhaustion of these cells (Horne-Debets et al., 2013). However, in contrast to the combined PD-L1/Lag-3 blockade study, no changes to T_{FH} numbers were observed. One likely explanation for this apparent contradiction is that PD-1 KO mice compared with WT mice had a significantly higher proportion of regulatory T follicular cells (T_{FR} cells) (Horne-Debets et al., 2013). T_{FR} cells are known to be suppressive in vitro and to limit the numbers of T_{FH} cells and GC B cells in vivo (Linterman et al., 2011). Alternatively, since PD-L1 can also interact specifically with B7-1 to inhibit T cell activation (Butte et al., 2007), this pathway may control T_{FH} numbers in PD-1 KO mice.

### CD8⁺ T Cells and Exhaustion

PD-1-mediated cellular exhaustion has been best associated with exhaustion of CD8⁺ T cells. However, as described earlier, a role for CD8⁺ T cells in the clearance of blood-stage malaria is not widely acknowledged although their role in pathogenesis of cerebral malaria and damage to splenic architecture (Beattie et al., 2006) are known. Critically, PD-1 was recently shown to mediate a 95% loss in the numbers and functional capacity of parasite-specific CD8⁺ T cells during the acute phase of malaria, which exacerbated the infection leading to chronic malaria (Horne-Debets et al., 2013). This study examined the progression of chronic malaria in PD-1 KO mice compared to wild type (WT) where 100% of mice develop chronic infections. Interestingly, <30% of the PD-1 KO mice developed chronic infections, and parasitemia levels in these mice were >100-fold lower than those in the WT mice. However, depletion of CD8⁺ T cells in PD-1 KO mice, increased peak parasitemia by 2-fold and 100% of the PD-1 KO mice developed chronic malaria (Horne-Debets et al., 2013). Overall, PD-1-mediated 80% reduction in numbers of tetramer⁺CD8⁺CD62L⁻ T cells and 95% reduction in capacity of CD8⁺ cells to proliferate in response to parasites, during the chronic phase of malaria (Horne-Debets et al., 2013). Of particular note is that even though PD-1 KO mice had more functional CD4⁺ T cells than WT mice and similar titers of parasite-specific antibodies, they still developed chronic malaria if CD8⁺ T cells were depleted.

Finally, PD-1 KO mice had more granzyme B-expressing CD8⁺ T cells than WT mice suggesting that cytotoxic-killing of infected cells was involved. These observations highlight the crucial role of CD8⁺ T cells in protection against chronic malaria. In contrast, a previous study had found blockade of PD-L1 augmented experimental cerebral malaria which is mediated by pathogenic CD8⁺ T cells (Hafalla et al., 2012), indicating the pathway protects against cerebral malaria. The clinical significance of these findings are highlighted by studies in Kenya which found human CD8⁺ T cells from individuals infected with malaria, express PD-1 (Illingworth et al., 2013). Thus the role of CD8⁺ T cells requires particular consideration as it may explain why despite years of exposure to intense Pf transmission there was no evidence of acquired, sterile immunity (Tran et al., 2013). It may be that antibodies and CD4⁺ T cells provide protection against symptomatic malaria but CD8⁺ T cells are required for sterile immunity. Thus with PD-1 mediated exhaustion of CD8⁺ T cells, sterile immunity is never acquired as recently reported (Tran et al., 2013).

### DC and Malaria

It is well established that during malaria, CD4+ T cells clear the primary peak parasitemia and B cells clear residual parasites. As T cell activation requires DCs, we compared DC function in five strains of mouse parasites and found a dichotomy in the phenotype and function of DCs between lethal and non-lethal strains and species of parasites (Wykes et al., 2007a; Wykes et al., 2007b) and as reviewed (Wykes and Good, 2008). These studies also found that DCs from infections with non-lethal P. yoelii 17XNL and P. chabaudi were fully functional and in particular secreted an abundance of IL-12 (Wykes et al., 2007a; Wykes et al., 2007b). By contrast, DCs from mice infected with three lethal strains of parasite, P. yoelii YM, P. vinckei and P. berghei, lacked functionality as they were unable to prime T cells or secrete IL-12 (Wykes et al., 2007a; Wykes et al., 2007b). When DCs from non-lethal P. yoelii 17XNL-infected mice were transferred to naive mice, recipient mice survived challenge with a lethal infection, and this effect was mediated by IL-12 (Wykes et al., 2007a). Moreover, other groups have also shown DC function to be compromised during malaria (Good et al., 2005; Ocana-Morgner et al., 2003; Urban et al., 1999; Urban et al., 2001).

PD-L2 is known to be predominantly expressed by DCs while PD-L1 is expressed on a range of cells including DCs. Thus, DCs from naive and infected mice were then examined for PD-L2 expression. PD-L2 mRNA levels were measured in DCs from naive mice and mice infected with non-lethal P. yoelii 17XNL or lethal P. yoelii YM (FIG. 2). DCs from lethal infections showed approximately 50% increase in PD-L2 mRNA while DCs from the non-lethal infection responded with nearly 300% increase, in protein expression. This study showed that higher PD-L2 expression correlated with better survival from malaria.

### Protective Role of PD-L2 During Malaria

To address whether PD-L2 expressed by DCs was protective, WT mice were infected with non-lethal malarias and treated with PD-L2-specific blocking antibodies to inhibit the function of this molecule. For this experiment, several cohorts of WT mice were infected with P. yoelii 17XNL and P. chabaudi and treated with either anti-PD-L2 or control rat IgG (Control Ig), 1 day after infection and every 3-4 days until days 14-18 p.i.

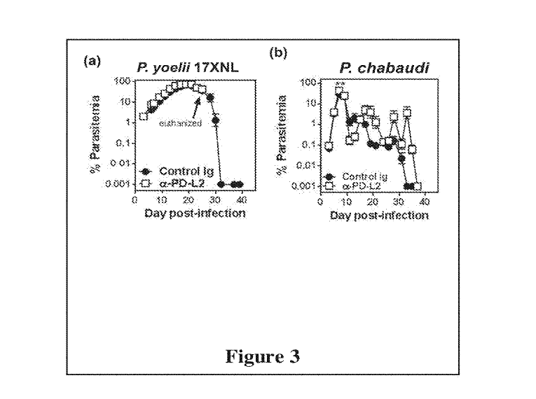

All WT mice receiving Control Ig (FIGS. 3a and b) cleared patent infections in 30-37 days. However, all mice given P. yoelii 17XNL and PD-L2 blocking antibodies died or were euthanized within 25 days, because of severe symptoms (FIG. 3a). In contrast, while all mice with chronic P. chabaudi malaria survived PD-L2 blockade, they had 16% higher primary peak parasitemia than control mice (note log scale; * denotes p=0.0048), higher parasitemia levels during the chronic phase of infection and took 4 days longer to clear the infection (FIG. 3b).

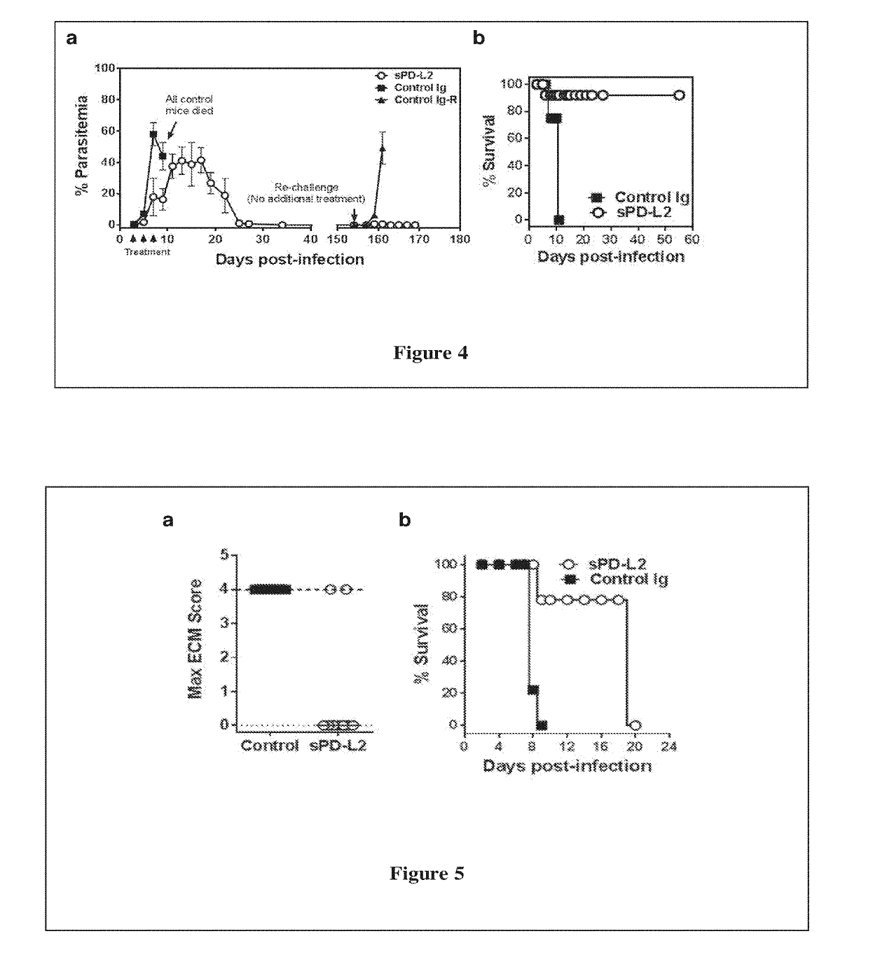

To determine if P. yoelii YM or P. berghei infections were lethal because of absent or low PD-L2 expression on DCs, these mice were supplemented sPD-L2 (FIGS. 4 and 5). For this, several cohorts of WT mice were infected with P. yoelii YM or P. berghei and on days 3, 5 and 7 post-infection were given soluble recombinant PD-L2-human-Fc synthetic protein. While all WT mice infected with P. yoelii YM and given control human IgG (Control Ig) died within 11 days, 92% of mice given multimeric sPD-L2 survived and cleared the infection in 25 days with a significantly lower peak parasitemia (FIGS. 4a and b; p<0.001). All surviving mice were then rested for 150 day and rechallenged with the same dose of lethal P. yoelii YM malaria (no additional PD-L2; FIG. 4a). All mice survived with minimal parasitemia (<1%) while all new control mice (Control Ig-R) succumbed to the infection. Interestingly, dimeric PD-L2 had a negative effect while higher multimers (e.g. octomeric sPD-L2) had a strong beneficial effect.

Analysis of mice with P. berghei malaria found that all control mice developed cerebral malaria symptoms (inc: ruffled fur, spasms, coma) within 8 days (FIG. 5a) and succumbed to the infection by day 10 (FIG. 5b). In contrast, all P. berghei-infected mice treated with sPD-L2 never developed cerebral symptoms, controlled the infection for approximately 15 days but all died on day 25 from uncontrolled parasitemia. Additional doses were not tested.

These studies confirmed that PD-L2 expression was required for immunity and survival from malaria. The blockade of PD-L2 in mice expressing this protein mediated lethality or exacerbated the infection. In contrast, if mice were supplemented with sPD-L2 when their DCs did not express PD-L2, they survived lethal infections or remained free of cerebral symptoms.

### PD-L2 Mediates Protection by CD4 T Cells

To determine if sPD-L2 improved immunity via T cells, P. yoelii YM-infected mice were given sPD-L2-in the absence or presence of CD4⁺ or CD8⁺ T cells. For this experiment, WT mice were given CD4⁺ or CD8⁺ T cell-depleting antibodies or treated with rat Ig (Rat Ig) 1 day before P. yoelii YM infections and every 3-4 days until days 14-18 i.p. Mice were then given sPD-L2 or Control human Ig, on day 3, 5 and 7 post-infection.

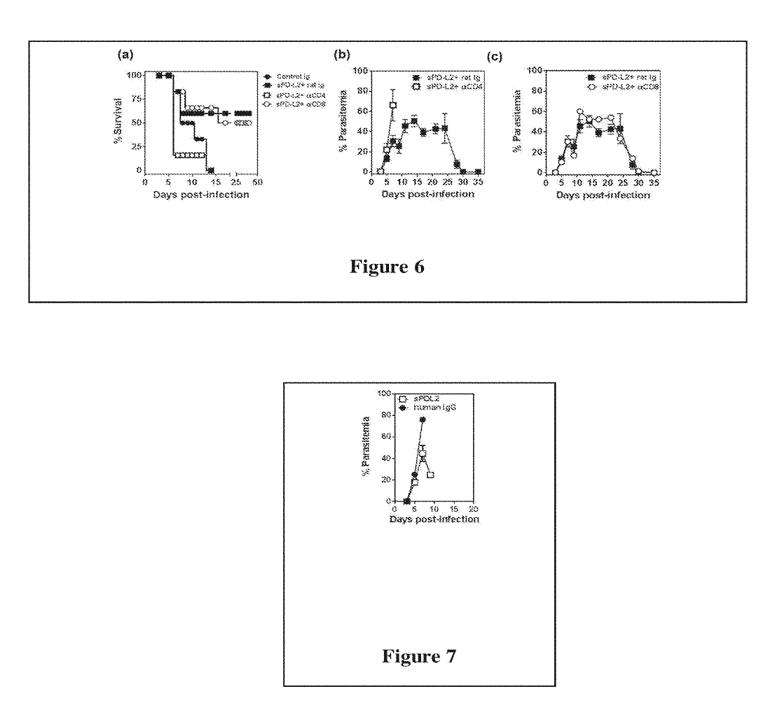

All WT mice receiving Control Ig (FIG. 6a) died or had to be euthanized by day 14. In contrast, 60% of P. yoelii YM-infected mice given sPD-L2 survived and cleared the infection in 30 days, However, if infected WT mice were given sPD-L2 but depleted of CD4⁺ T cells, all mice died or had to be euthanized (FIGS. 6a and b) because they developed severe clinical symptoms. In contrast, depletion of CD8⁺ T cells did not affect survival from lethal malaria by sPD-L2 treatment but mice did experience higher parasitemias (FIG. 6c). Taken together, these observations demonstrated that sPD-L2 mediated protection and survival by CD4⁺ T cells with some effect on CD8+ T cell function. These studies were undertaken in very young mice (due to the lack of availability of mature mice) where median survival following sPD-L2 treatment was lower than in adult mice.

Protection by sPD-L2 is not Mediated by Blockade of PD-L1 Function.

Given that expression of PD-L2 on DCs or sPD-L2 treatment could mediate protective immunity, we hypothesized that PD-L2 may mediate protection by blockade of PD-L1-mediated inhibition of immunity. To test this hypothesis, two cohorts of PD-L1 knockout mice (PD-L1KO; n=4) were infected with P. yoelii YM and treated with either three doses of PD-L2 or control IgG. All control mice died with severe clinical symptoms and high parasitemia levels (˜78%) by day 7 (FIG. 7). In contrast, infected PD-L1KO mice treated with PD-L2 controlled parasitemia (24%) but died by day 10. This study indicated protection mediated by PD-L2 was independent of PD-L1.

### Galectin-9 on CD4+ T Cells is a Novel Receptor for PD-L2.

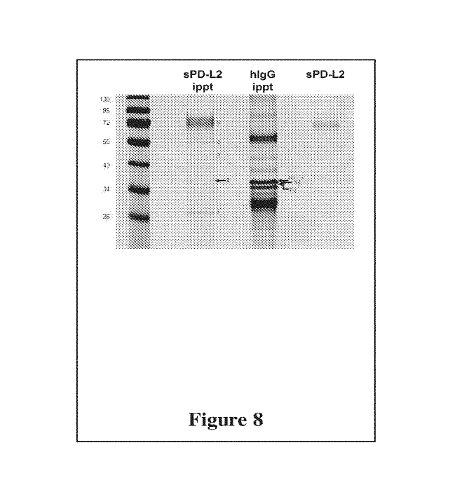

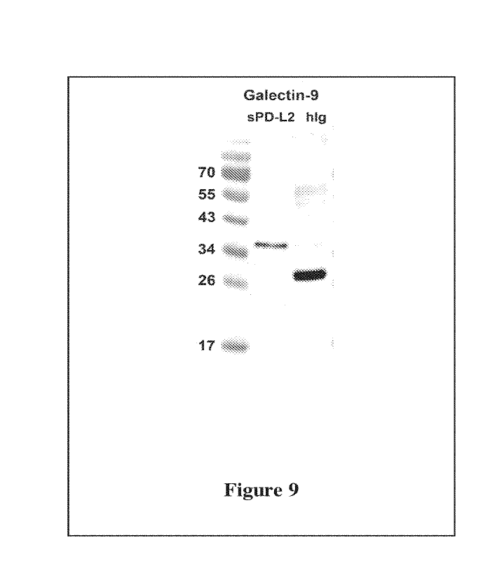

Given that PD-L2 was protective against malaria, independent of PD-L1, we hypothesized it had a second receptor on naive T cells. To test this hypothesis, we prepared lysates of isolated T cells from naive C57BL/6 mice and used immobilized PD-L2 or human IgG to immuno-precipitate the receptor (FIG. 8). Five reproducible bands were repeatedly immuno-precipitated in 3 independent experiments, included (heavy and light immunoglobulin chains (bands 1 and 4), sPD-L2 (band 5) and actin (band 3). Galectin-9 (band 2) was immunoprecipitated by PD-L2 but did not have an equivalent band immunoprecipitated by human IgG in 3 independent experiments. The band designated N2° and N2 in the control were histone proteins. Finally, to confirm that band 2 immuno-precipitated by sPD-L2 was Galectin-9, the experiment was repeated but the gel transferred to nitrocellulose and the Western blot labelled with an anti-galectin-9 antibody (FIG. 9). These studies confirmed that the 39 kD band found by sequencing of the band 2 immuno-precipitated from T cells was Galectin-9 and was potentially a novel binding partner for PD-L2.

### Galectin-9 on Naive T Cells is a Receptor for PD-L2.

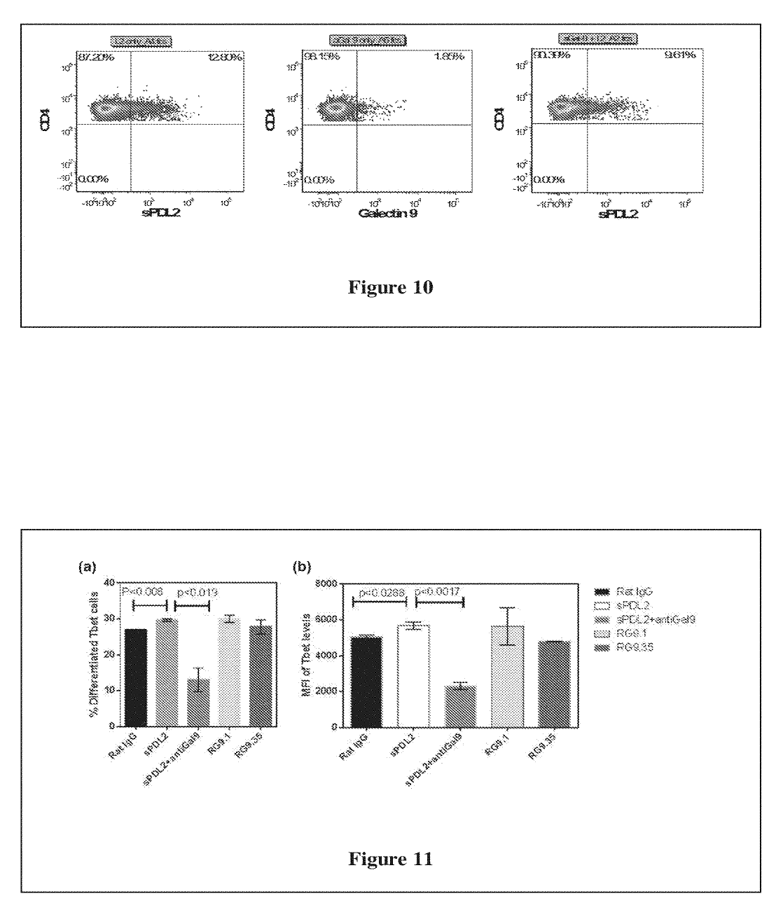

To determine if sPD-L2 bound Galectin-9 on intact T cells, total T cell populations were isolated from spleens of naive mice and incubated with bitionylated sPD-L2 and APC-Streptavidin or PE-anti-galectin-9 (FIG. 10). Flowcytometry analysis found that while sPD-L2 bound approximately 12.8% of naive CD4+ T cells, galectin-9 was only expressed on approximately 1.9% of these cells. Pre-treatment of naive T cells with excess, unlabelled anti-galectin-9 antibody, reduced labelling by sPD-L2 by approximately 3% confirming sPD-L2 bound galectin-9 on T cells. Previous published studies showed that approximately 10-20% of CD4+ T cells taken from the spleen of naive mice express PD-1 which could bind PD-L2. Finally, sPD-L2 did not bind significant numbers of CD8+ T cells in this assay.

sPD-L2 and Anti-Galectin-9 Mediate Survival and Differentiation in Naive CD4+ & CD8+ T Cells

T cells isolated from naive mice were cultured in 96 well plates coated with anti-CD3 (5 μg/ml) to provide antigen signals along with IL-2. The cultures were also supplemented with either (a) plate bound rat IgG as a control, (b) plate bound sPD-L2; (c) plate bound sPD-L2 and cells treated with a Galectin-9 inhibitor in the form of the anti-galectin-9 (clone 108A) antibody (d) a Galectin-9 agonist antibody (clone RG9.1) and (e) anti-galectin-9 (clone RG9.35). sPD-L2 increased the percentage of CD4+CD62L^{lo} cells which expressed T_{BET} (FIGS. 11a) and (b) the level of T_{BET} within cells compared to rat IgG control (FIG. 11b). This effect was blocked by anti-Galectin-9 (clone 108A) antibody. Clone RG9.1 also increased the percentage of CD4+CD62L^{lo} cells which expressed T_{BET} and the level of T_{BET} within cells (FIGS. 11a and b).

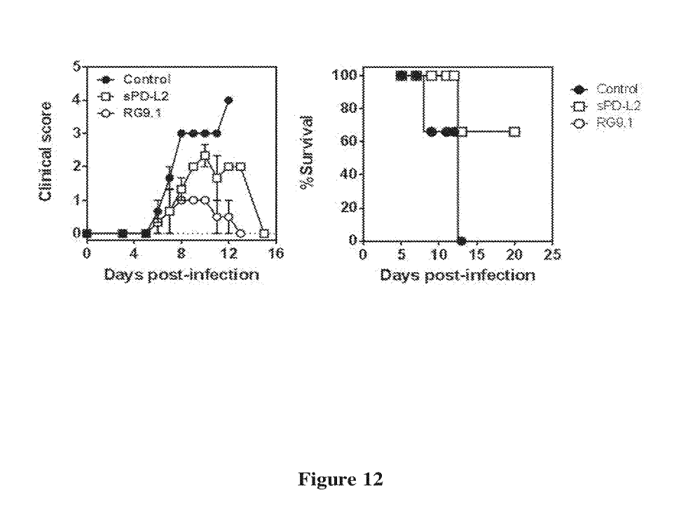

The viability of cells treated with plate bound PD-L2 and RG.1 (RG9.1) was higher than other cultures after 36 hours so some of these culture experiments were repeated with 72 hour cultures with a much lower CD3 level stimulation (1 μg/ml). As seen in FIG. 12, sPD-L2 and RG1 (RG9.1) antibody both improve viability of CD4+ and CD8+ T cells compared to control cultures.

### Soluble PD-L2 and Anti Galectin-9 Antibody Protect Against Lethal Malaria

To determine if signalling Galectin-9 has the same effects as soluble PD-L2, three cohorts of WT mice were infected with P. yoelii YM and on days 3, 5 and 7 post-infection were given 200 μg sPD-L2, anti-galectin-9 or rat IgG intravenously. Mice were monitored daily and scored for clinical symptoms of disease including ruffled fur, hunching or lack of activity. Mice given sPD-L2 or an agonising anti-galectin 9 (clone RG9.1) showed minimal symptoms and ⅔ mice in these groups survived when all control mice had died or been euthanized (FIG. 12).

Galectin-9 is a Binding Partner for sPD-L2

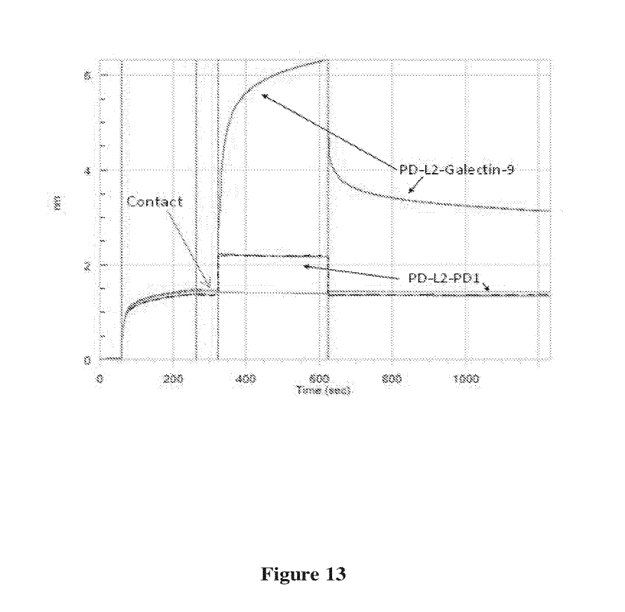

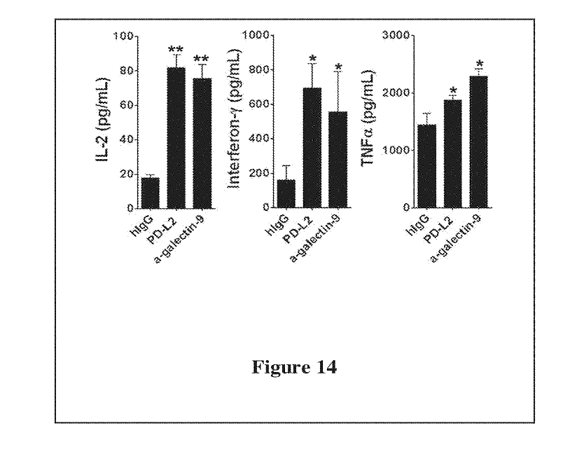

Octet red studies were undertaken to determine the biochemical nature of binding between mouse Galectin-9 and mouse PD-L2. The sPD-L2 was bound to the probe and its interaction with sPD-1 and sGalectin-9 measured. As shown in FIG. 13, the results show an almost instantaneous association and dissociation between PD-L2 and PD-1. In contrast, Galectin-9 binding takes ˜200 sec to associate with PD-L2 and >614 sec to dissociate indicating a very stable interaction. Most significantly, while the PD-L2-PD-1 interaction is at a molecule ratio of 1:1, the Galectin-9 and PD-L2 interaction involves aggregation or multimerisation of Galectin-9 during binding. This helps to explain why a multimeric form of sPD-L2 is protective against malaria while the monomeric or dimeric form may not protect. Our study comparing protection by monomeric and multimeric sPD-L2 found monomeric sPD-L2 did not protect mice from lethal P. yoelii YM malaria (n=4) or prevent cerebral malaria (n=3) compared to multimeric sPD-L2 where 77-92% of mice were protected (FIG. 4). Furthermore, monomeric sPD-L2 exacerbated cerebral malaria suggesting it blocked sPD-L2's interaction with Galectin-9. As such, the form of sPD-L2 applied can be used to control the nature of the immune response, (e.g. multimeric forms can be administered to protect against malaria or cancer and the monomeric form administered to down-regulate the immune system to treat inflammatory or autoimmune diseases such as asthma or Chrohn's disease). In this regard, agents that promote multimerisation of PD-L2 in vivo are also useful in the invention such as the use of aptamers and bispecific antibodies. Aptamers are small oligonucleotides that can specifically bind to a wide range of target molecules and offer some advantages over antibodies as therapeutic agents. These could mimic multimeric PD-L2.

### Anti-Galectin-9 Antibody Activates Mouse CD4⁺ T Cells in Culture to Secrete Th1 Cytokines

Galectin-9 expressed by DCs is also a ligand for TIM-3 on T cells (Zhu et al., 2005). Soluble galectin-9-induced death of Th1 cells was dependent on TIM-3—in vitro, and administration of galectin-9 protein in vivo resulted in selective loss of interferon-gamma-producing T cells (Zhu et al., 2005). The role of Galectin-9 expressed by T cells is less clear. We initially tested 3 anti-galectin-9 antibodies and found ⅔ were activating (data not shown). We analysed the effect of the co-stimulatory anti-galectin-9 antibody with strongest stimulatory activity on purified T cells in vitro. We evaluated the effects of control IgG, soluble mouse PD-L2-Ig or anti-mouse galectin-9 mAb on CD4⁺ T cells isolated from mouse spleens and cultured on plastic plates coated with anti-CD3 antibody. After 3 days of culture, supernatants were tested for cytokines. Compared to control cultures with IgG, both immobilized PD-L2 and anti-galectin-9 were able to significantly increase IL-2 (˜4-fold), IFN-γ (˜4-fold) and TNF-α (60%) secretion. These studies confirmed that both PD-L2 and anti-galectin-9 were providing mouse T cells with co-stimulatory signals to improve Th1 responses as reported previously for mouse PD-L2 (Shin et al., 2003).

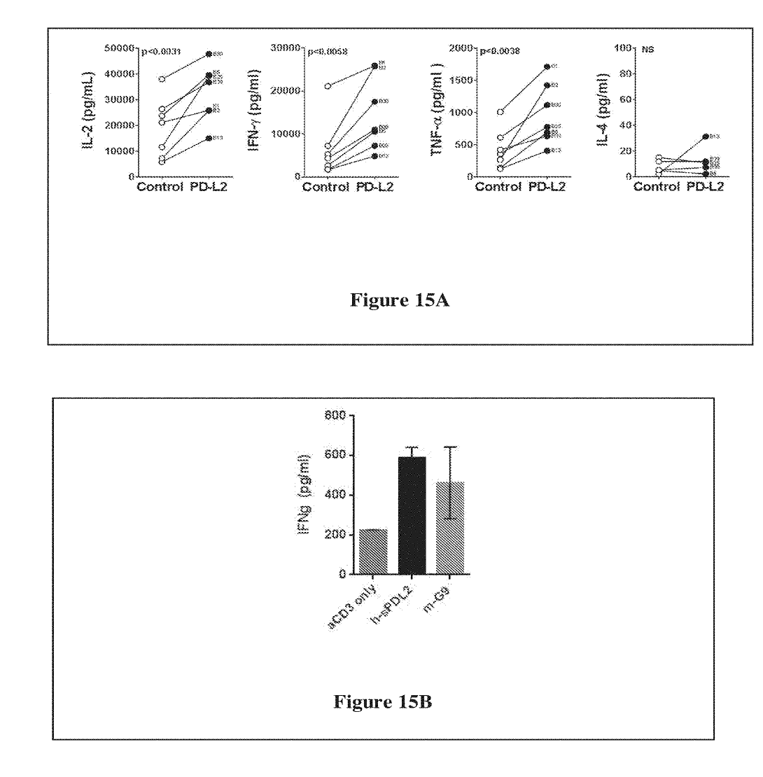

A similar assay undertaken for human CD4⁺ T cells also showed sPD-L2 could increase secretion of Th1 cytokines (FIG. 15A).

As we could not find an anti-human Galectin-9 antibody that stimulates cytokine production by human CD4+ T cells, we tested an anti-mouse Galectin-9 antibody (FIG. 15B). This anti-mouse Galectin-9 antibody induced secretion of interferon-γ (but not other cytokines) to levels induced by human sPD-L2.

### Anti-Galectin-9 Protects Against Malaria

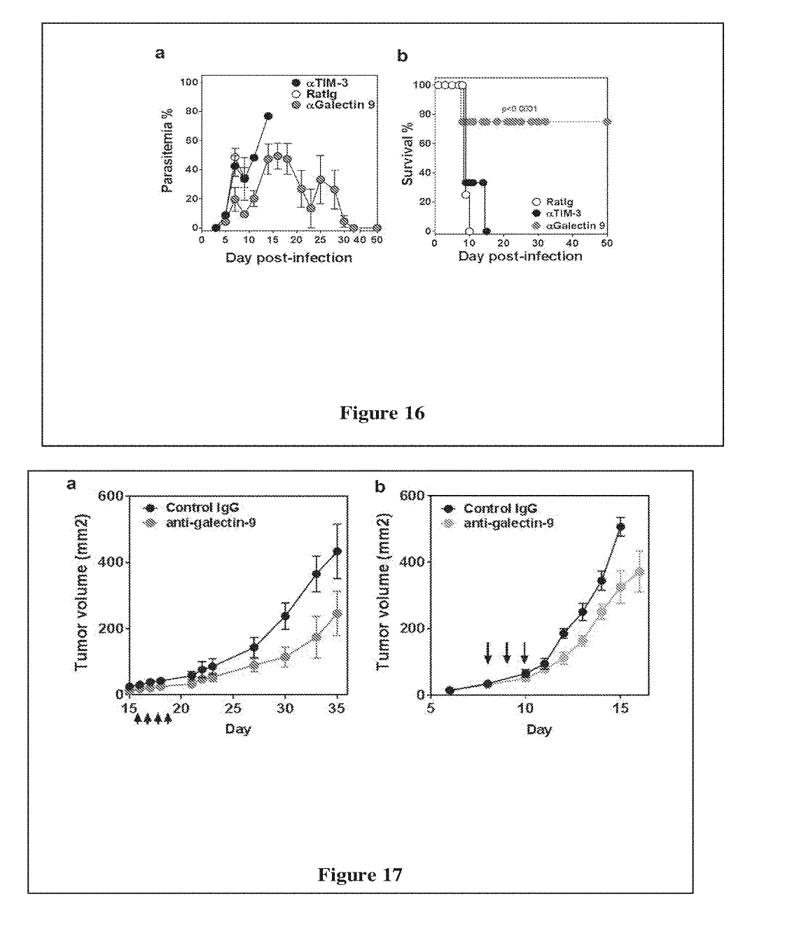

To confirm that the anti-galectin-9 antibody was capable of providing the same protection seen by treatment with soluble PD-L2 (FIG. 4), WT mice were infected with lethal P. yoelii YM and treated with either control rat Ig, blocking anti-Tim-3, or anti-mouse galectin-9 (FIGS. 16 a and b). In replicate experiments, 3 doses of the anti-galectin-9 antibody mediated survival for 75% of mice compared to no protection offered by antibody-mediated blockade of TIM-3, another receptor for galectin-9. Tim-3 blockade offered no significant protection compared to control rat Ig treated mice.

### Anti-Galectin-9 Reduces Tumour Progression

Given that Th1 CD4⁺ T cell immunity is also vital for clearing tumours, activating anti-galectin-9 antibody was then tested in two syngeneic mouse breast cancer models. Four doses of anti-galectin-9 antibody could retard growth of a palpable PYMT-derived mammary carcinoma, orthotopically injected into the fourth left mammary fat pad of each recipient mouse (FIG. 17a). The anti-galectin-9 treatment given on days 16-22 slowed tumour progression between days 27 and 35 compared to the isotype control group. A previous study had investigated if Treg cell ablation combined with CTLA-4 or PD-1/PD-L1 blockade affected the same orthotopically implanted PYMT-driven mammary carcinoma (Bos et al., 2013). It was found that while Treg cell ablation significantly delayed primary and metastatic tumour progression, checkpoint blockade did not affect oncogene-driven tumour growth. We then tested if three doses of anti-galectin-9, given Days 8-10, could retard growth of an aggressive metastatic EO771.LMB mammary adenocarcinoma (Johnstone et al., 2015). While all control mice were euthanized by day 15, treated mice had a 35% smaller tumours on day 15-16 compared to Day 15 controls (FIG. 17b). Overall, activating anti-galectin-9 treatment reduced progression of orthotopically implanted mammary carcinomas.

### Blockade of PD-L2 can Inhibit Tbet+ Th1 Responses

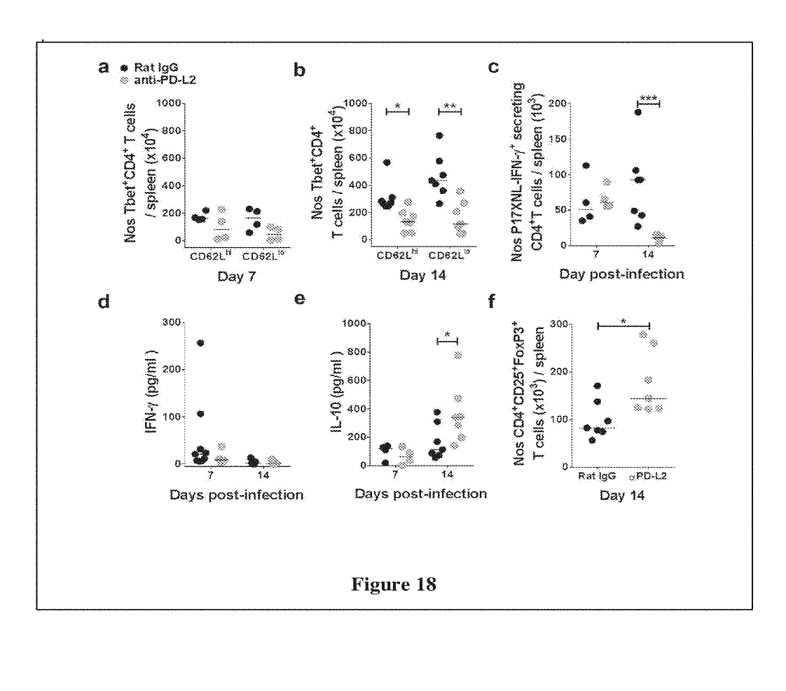

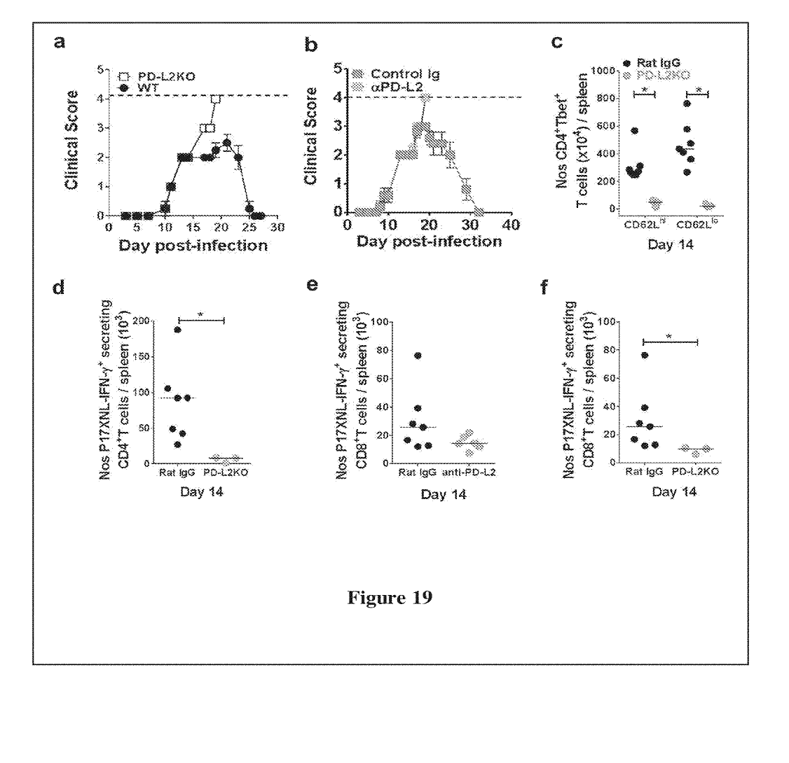

To confirm that PD-L2 did control Tbet and Th1 immunity in vivo, we infected mice with P. yoelii 17XNL malaria and blocked PD-L2 with a monoclonal antibody when parasites became detectable in the blood. For this experiment, WT mice were infected with P. yoelii 17XNL and given either an anti-PD-L2 or control rat IgG, 4 days post-infection (p.i.) and every 3-4 days until day 14-18 p.i. First, CD4⁺ T cells were examined for the expression of Tbet, a transcription factor required for effector functions of Th1 CD4⁺ T cells, which are known to mediate protection against malaria (Ing and Stevenson, 2009; Stephens and Langhorne, 2010). T cells were also evaluated for expression of CD62L, a marker found on naive T cells and which also distinguishes central memory (CD62L^{hi}) from effector memory (CD62L^{lo} T cells. There was a trend for lowered numbers of splenic Tbet-expressing CD4⁺ T cells with PD-L2 blockade after 7 days of infection (FIG. 18a). By day 14, however, the control mice had 2.2 and 3-fold more Tbet-expressing CD62L^{hi} and CD62L^{lo} CD4⁺ T cells per spleen, respectively, than the mice with blockade of PD-L2 (FIG. 18b). Similarly, control mice had >5-fold higher numbers of IFN-γ-secreting, parasite-specific CD4⁺ T cells, as measured by an ELISPOT assay at day 14, than mice with PD-L2 blockade (FIG. 18c). Levels of serum IFN-γ, which can be secreted by several cell types, was reduced at day 7 in the mice with PD-L2 blockade, and was low in both groups of mice by day 14 (FIG. 18d). In contrast, mice with PD-L2 blockade had >2-fold more serum IL-10 than control mice by day 14 (FIG. 18e). This result correlates with the 2.6-fold higher number of regulatory T cells (T_{REG}) per spleen (FIG. 18f). Studies with P. yoelii 17XNL-infected PD-L2KO mice also found significantly lower numbers of Tbet-expressing and IFN-γ-secreting, parasite-specific CD4⁺ T cells per spleen at day 14 compared to infected WT mice (FIG. 19c, d). Finally, with either PD-L2KO mice or PD-L2 blockade with antibodies, there was a trend towards lower numbers of IFN-γ-secreting, parasite-specific CD8⁺ T cells by day 14 of infection (FIG. 19e, f).

Taken together, our data showed that PD-L2 expression was necessary for effective Th1 CD4⁺ T cell responses during P. yoelii 17XNL malaria infection. Significantly, PD-L2 was required for the optimal expansion of Tbet-expressing CD4⁺ T cells, as blockade of PD-L2 prevented an increase in the number of these cells between days 7 and 14 of a P. yoelii 17XNL infection. Of note, functional, parasite-specific IFN-γ-secreting CD4⁺ T cells were present at day 7 but reduced by day 14 in the absence of PD-L2 signals indicating this signal improved expansion and survival of these key effector cells. In light of these findings, P. chabaudi-infected mice most likely survived PD-L2-blockade, as the bulk of parasites are cleared within 10 days, but the P. yoelii 17XNL experiments showed PD-L2 only improves longer-term immunity after the first week. Thus, PD-L2 is required to sustain Th1 CD4⁺ T cell numbers only after the first week of infection.

The T-box transcription factor T-bet has emerged as a key regulator of type 1-like immunity, playing critical roles in the establishment and/or maintenance of effector cell fates in T and B lymphocytes, as well as dendritic cells and natural killer cells. T-bet likely plays a critical role in the maintenance of Th1 effector function. T-bet-deficient mice demonstrate impaired Th1 differentiation, including defective IFN-γ production primarily in CD4 and γδ T cells.

Th1 responses have long been associated with autoimmune syndromes. Both Celiac and Crohn's diseases, which have generally been considered Th1-related syndromes, exhibit enhanced T-bet activity and/or expression. In a Th1-related IBD mouse model, adoptive transfer of CD4+CD62L+ cells into severe combined immunodeficient (scid) recipients showed that T-bet deficiency protects against, while T-bet overexpression promotes, disease. This is also the case for multiple sclerosis (Rack et al. 2014), inflammatory arthritis (Wang et al, 2006,), diabetes (Juedes et al, 2004,), Behçet's syndrome (Li et al, 2006 T), and VogtKoyanagi-Harada (VKH) disease (Li et al., 2005). Since blocking the PD-L2/galectin-9 pathway blocks Tbet, it therefore has potential to provide a new method for treatment of autoimmune diseases.

sPD-L2-Mediated Survival from Lethal Malaria Requires CD4⁺ T Cells

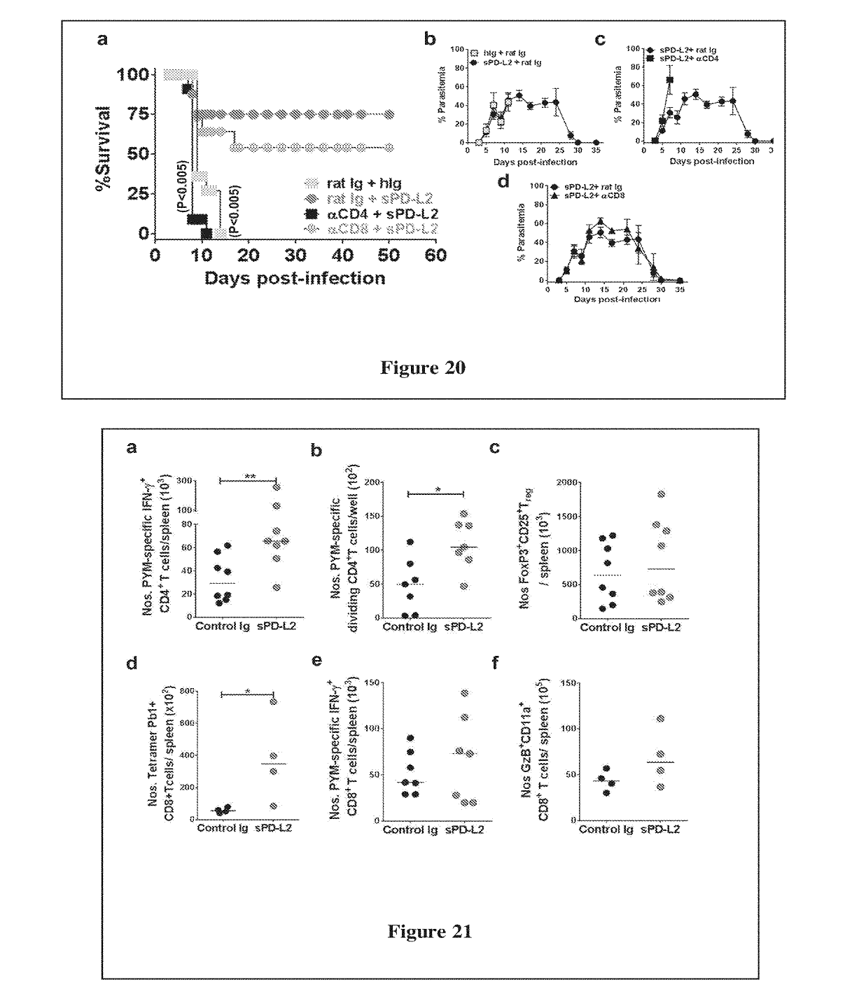

To determine the contribution of T cells to multimeric sPD-L2-mediated survival from P. yoelii YM malaria, CD4⁺ or CD8⁺ T cells were depleted in sPD-L2-treated, infected mice. For this experiment, multiple groups of WT mice were infected with P. yoelii YM and treated with sPD-L2 or human IgG (hIg). These mice were also given CD4⁺ or CD8⁺ T cell-depleting antibodies or rat Ig on day 1 and every 3-4 days until day 14-18 p.i. Previous studies confirmed that the antibodies used would deplete these cells. All of the infected WT mice that received hIg and rat Ig died or required euthanasia by day 14 (FIGS. 20a and b). In contrast, 75% of the P. yoelii YM-infected mice given sPD-L2 and control rat Ig cleared parasitemia within 30 days and survived >50 days, when monitoring was stopped (FIGS. 6a and b). However, mice were not protected by sPD-L2 if the CD4⁺ T cells were depleted and had to be euthanized due to severity of clinical symptoms (FIG. 20a, c). In contrast, depletion of CD8⁺ T cells did not significantly affect the protective effect provided by sPD-L2, although these mice had consistently higher parasitemia around days 11-21 than control mice (FIG. 20a, d). Taken together, these findings demonstrate that sPD-L2 can promote protection, survival, and parasite control from P. yoelii YM infection through CD4⁺ T cells with a possible minor contribution from CD8⁺ T cells.

sPD-L2 Mediates Protection and Survival Through Improved CD4⁺ and CD8⁺ T Cell Function

To determine the mechanism by which sPD-L2 exerts its therapeutic effects, P. yoelii YM-infected mice were treated with control Ig or sPD-L2 on days 3 and 5, and the spleens were collected at day 7 before the onset of severe clinical symptoms in the control mice. T cells were isolated from spleens and cultured with spleen DCs from naive mice and parasite-specific antigen (MSP1₁₉) or peptide (Pb1, SQLLNAKYL) or no additional antigen. Treatment with sPD-L2 increased the number of parasite-specific CD4⁺ T cells that could respond to MSP1₁₉ in culture, with ˜2.7-fold higher numbers of IFN-γ secreting CD4⁺ T cells than the mice treated with control Ig, as measured by an ELISPOT assay (FIG. 21a). Similarly, an in vitro EdU-uptake assay confirmed that sPD-L2-treated mice had higher numbers of parasite-specific T cells which proliferated in response to parasite antigen (FIG. 21b). However, there was no difference in the number of T_{REGs} between cohorts (FIG. 21c). Furthermore, the sPD-L2-treated mice also exhibited 6-fold higher numbers of parasite-specific CD8⁺ T cells (i.e. that bound MHC tetramer (D^{b}) displaying the parasite-specific peptide F4 (Lau et al., 2011)) than the control group (FIG. 21d). However, there was no significant increase in IFN-γ secretion (FIG. 21e) or granzyme B expression (FIG. 21f) by these cells within 7 days. Taken together, these results show that sPD-L2 protects mice from lethal malaria by promoting development of IFN-γ secreting CD4⁺ T cells which indicates improved Th1 effector functions known to be crucial for protection against malaria (Kumar and Miller, 1990; Stephens and Langhorne, 2010; Su and Stevenson, 2002). Similarly, the increased CD8+ T cells explained the modest improvement in protection seen in mice treated with sPD-L2 around days 11 to 21 (FIG. 18d).

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to herein is incorporated herein by reference in their entirety.

### REFERENCES

Agata, Y., A. Kawasaki, H. Nishimura, Y. Ishida, T. Tsubata, H. Yagita, and T. Honjo. 1996. Expression of the PD-1 antigen on the surface of stimulated mouse T and B lymphocytes. Int Immunol 8:765-772.
Akpogheneta, O. J., N. O. Duah, K. K. Tetteh, S. Dunyo, D. E. Lanar, M. Pinder, and D. J. Conway. 2008. Duration of naturally acquired antibody responses to blood-stage Plasmodium falciparum is age dependent and antigen specific. Infect Immun 76:1748-1755.
Beattie, L., C. R. Engwerda, M. Wykes, and M. F. Good. 2006. CD8+ T lymphocyte-mediated loss of marginal metallophilic macrophages following infection with Plasmodium chabaudi chabaudi AS. J Immunol 177:2518-2526.
Bos, P. D., Plitas, G., Rudra, D., Lee, S. Y., and Rudensky, A. Y. (2013). Transient regulatory T cell ablation deters oncogene-driven breast cancer and enhances radiotherapy. J Exp Med 210, 2435-2466.
Butler, N. S., J. Moebius, L. L. Pewe, B. Traore, O. K. Doumbo, L. T. Tygrett, T. J. Waldschmidt, P. D. Crompton, and J. T. Harty. 2012. Therapeutic blockade of PD-L1 and LAG-3 rapidly clears established blood-stage Plasmodium infection. Nat Immunol 13:188-195.
Butte, M. J., M. E. Keir, T. B. Phamduy, A. H. Sharpe, and G. J. Freeman. 2007. Programmed death-1 ligand 1 interacts specifically with the B7-1 costimulatory molecule to inhibit T cell responses. Immunity 27:111-122.
Chandele, A., P. Mukerjee, G. Das, R. Ahmed, and V. S. Chauhan. 2011. Phenotypic and functional profiling of malaria-induced CD8 and CD4 T cells during blood-stage infection with Plasmodium yoelii. Immunology 132:273-286.
Chemnitz, J. M., R. V. Parry, K. E. Nichols, C. H. June, and J. L. Riley. 2004. SHP-1 and SHP-2 associate with immunoreceptor tyrosine-based switch motif of programmed death 1 upon primary human T cell stimulation, but only receptor ligation prevents T cell activation. J Immunol 173:945-954.
Crompton, P. D., M. A. Kayala, B. Traore, K. Kayentao, A. Ongoiba, G. E. Weiss, D. M. Molina, C. R. Burk, M. Waisberg, A. Jasinskas, X. Tan, S. Doumbo, D. Doumtabe, Y. Kone, D. L. Narum, X. Liang, O. K. Doumbo, L. H. Miller, D. L. Doolan, P. Baldi, P. L. Felgner, and S. K. Pierce. 2010. A prospective analysis of the Ab response to Plasmodium falciparum before and after a malaria season by protein microarray. Proc Natl Acad Sci USA 107:6958-6963.
Dong, H., G. Zhu, K. Tamada, and L. Chen. 1999. B7-H1, a third member of the B7 family, co-stimulates T-cell proliferation and interleukin-10 secretion. Nat Med 5:1365-1369.
Dorfman, J. R., P. Bejon, F. M. Ndungu, J. Langhorne, M. M. Kortok, B. S. Lowe, T. W. Mwangi, T. N. Williams, and K. Marsh. 2005. B Cell Memory to 3 Plasmodium falciparum Blood-Stage Antigens in a Malaria-Endemic Area. J Infect Dis 191:1623-1630.
Egan, A. F., J. A. Chappel, P. A. Burghaus, J. S. Morris, J. S. McBride, A. A. Holder, D. C. Kaslow, and E. M. Riley. 1995. Serum antibodies from malaria-exposed people recognize conserved epitopes formed by the two epidermal growth factor motifs of MSP1(19), the carboxy-terminal fragment of the major merozoite surface protein of Plasmodium falciparum. Infect Immun 63:456-466.
Egan, A. F., J. Morris, G. Barnish, S. Allen, B. M. Greenwood, D. C. Kaslow, A. A. Holder, E. M. Riley, J. A. Chappel, P. A. Burghaus, J. S. Morris, and J. S. McBride. 1996. Clinical immunity to Plasmodium falciparum malaria is associated with serum antibodies to the 19-kDa C-terminal fragment of the merozoite surface antigen, PfMSP-1. J Infect Dis 173:765-769.
Freeman, G. J., A. J. Long, Y. Iwai, K. Bourque, T. Chernova, H. Nishimura, L. J. Fitz, N. Malenkovich, T. Okazaki, M. C. Byrne, H. F. Horton, L. Fouser, L. Carter, V. Ling, M. R. Bowman, B. M. Carreno, M. Collins, C. R. Wood, and T. Honjo. 2000. Engagement of the PD-1 immunoinhibitory receptor by a novel B7 family member leads to negative regulation of lymphocyte activation. J Exp Med 192:1027-1034.
Good, M. F., H. Xu, M. Wykes, and C. R. Engwerda. 2005. Development and regulation of cell-mediated immune responses to the blood stages of malaria: implications for vaccine research. Annu Rev Immunol 23:69-99.
Hafalla, J. C., C. Claser, K. N. Couper, G. E. Grau, L. Renia, J. B. de Souza, and E. M. Riley. 2012. The CTLA-4 and PD-1/PD-L1 Inhibitory Pathways Independently Regulate Host Resistance to Plasmodium-induced Acute Immune Pathology. PLoS Pathog 8:e1002504.
Horne-Debets, J. M., R. Faleiro, D. S. Karunarathne, X. Q. Liu, K. E. Lineburg, C. M. Poh, G. M. Grotenbreg, G. R. Hill, K. P. Macdonald, M. F. Good, L. Renia, R. Ahmed, A. H. Sharpe, and M. N. Wykes. 2013. PD-1 Dependent Exhaustion of CD8(+) T Cells Drives Chronic Malaria. Cell Reports 5:1204-1213.
Illingworth, J., N. S. Butler, S. Roetynck, J. Mwacharo, S. K. Pierce, P. Bejon, P. D. Crompton, K. Marsh, and F. M. Ndungu. 2013. Chronic exposure to Plasmodium falciparum is associated with phenotypic evidence of B and T cell exhaustion. J Immunol 190:1038-1047.
Ing, R., and Stevenson, M. M. (2009). Dendritic cell and NK cell reciprocal cross talk promotes gamma interferon-dependent immunity to blood-stage Plasmodium chabaudi AS infection in mice. Infect Immun 77, 770-782.
Iwai, Y., S. Terawaki, M. Ikegawa, T. Okazaki, and T. Honjo. 2003. PD-1 inhibits antiviral immunity at the effector phase in the liver. J Exp Med 198:39-50.
Jacobs, P., D. Radzioch, and M. M. Stevenson. 1996. A Th1-associated increase in tumor necrosis factor alpha expression in the spleen correlates with resistance to blood-stage malaria in mice. Infect Immun 64:535-541.
Johnstone, C. N., Smith, Y. E., Cao, Y., Burrows, A. D., Cross, R. S., Ling, X., Redvers, R. P., Doherty, J. P., Eckhardt, B. L., Natoli, A. L., et al. (2015). Functional and molecular characterisation of EO771.LMB tumours, a new C57BL/6-mouse-derived model of spontaneously metastatic mammary cancer. Disease models & mechanisms 8, 237-251.
Juedes et al, 2004, T-bet Controls Autoaggressive CD8 Lymphocyte Responses in Type 1 Diabetes. J Exp Med. 2004 Apr. 19; 199(8): 1153-1162
Keir, M. E., M. J. Butte, G. J. Freeman, and A. H. Sharpe. 2008. PD-1 and its ligands in tolerance and immunity. Annu Rev Immunol 26:677-704.
Kinyanjui, S. M., D. J. Conway, D. E. Lanar, and K. Marsh. 2007. IgG antibody responses to Plasmodium falciparum merozoite antigens in Kenyan children have a short half-life. Malar J 6:82.
Kumar, S., and Miller, L. H. (1990). Cellular mechanisms in immunity to blood stage infection. Immunol Lett 25, 109-114.
Latchman, Y., C. R. Wood, T. Chernova, D. Chaudhary, M. Borde, I. Chernova, Y. Iwai, A. J. Long, J. A. Brown, R. Nunes, E. A. Greenfield, K. Bourque, V. A. Boussiotis, L. L. Carter, B. M. Carreno, N. Malenkovich, H. Nishimura, T. Okazaki, T. Honjo, A. H. Sharpe, and G. J. Freeman. 2001. PD-L2 is a second ligand for PD-1 and inhibits T cell activation. Nat Immunol 2:261-268.
Lau, L. S., Fernandez Ruiz, D., Davey, G. M., de Koning-Ward, T. F., Papenfuss, A. T., Carbone, F. R., Brooks, A. G., Crabb, B. S., and Heath, W. R. (2011). Blood-stage Plasmodium berghei infection generates a potent, specific CD8+ T-cell response despite residence largely in cells lacking MEW I processing machinery. J Infect Dis 204, 1989-1996.
Li B et al, T-bet expression is upregulated in active Behçet's disease. Br J Ophthalmol. 2003 October; 87(10):1264-7
Li B et al., Upregulation of T-bet expression in peripheral blood mononuclear cells during Vogt-Koyanagi-Harada disease. Br J Ophthalmol. 2005 November; 89(11):1410-2.
Linterman, M. A., W. Pierson, S. K. Lee, A. Kallies, S. Kawamoto, T. F. Rayner, M. Srivastava, D. P. Divekar, L. Beaton, J. J. Hogan, S. Fagarasan, A. Liston, K. G. Smith, and C. G. Vinuesa. 2011. Foxp3+ follicular regulatory T cells control the germinal center response. Nat Med 17:975-982.
McCall, M. B., J. Hopman, M. Daou, B. Maiga, V. Dara, I. Ploemen, K. Nganou-Makamdop, A. Niangaly, Y. Tolo, C. Arama, J. T. Bousema, J. W. van der Meer, A. J. van der Ven, M. Troye-Blomberg, A. Dolo, O. K. Doumbo, and R. W. Sauerwein. 2010. Early interferon-gamma response against Plasmodium falciparum correlates with interethnic differences in susceptibility to parasitemia between sympatric Fulani and Dogon in Mali. J Infect Dis 201:142-152.
McCall, M. B., and R. W. Sauerwein. 2010. Interferon-gamma—central mediator of protective immune responses against the pre-erythrocytic and blood stage of malaria. J Leukoc Biol 88:1131-1143.
Muxel, S. M., A. P. Freitas do Rosario, C. A. Zago, S. I. Castillo-Mendez, L. R. Sardinha, S. M. Rodriguez-Malaga, N. O. Camara, J. M. Alvarez, and M. R. Lima. 2011. The spleen CD4+ T cell response to blood-stage Plasmodium chabaudi malaria develops in two phases characterized by different properties. PLoS One 6:e22434.
Ocana-Morgner, C., M. M. Mota, and A. Rodriguez. 2003. Malaria blood stage suppression of liver stage immunity by dendritic cells. J Exp Med 197:143-151.
Sharpe, A. H., E. J. Wherry, R. Ahmed, and G. J. Freeman. 2007. The function of programmed cell death 1 and its ligands in regulating autoimmunity and infection. Nat Immunol 8:239-245.
Shin, T., Kennedy, G., Gorski, K., Tsuchiya, H., Koseki, H., Azuma, M., Yagita, H., Chen, L., Powell, J., Pardoll, D., and Housseau, F. 2003. Cooperative B7-1/2 (CD80/CD86) and B7-DC costimulation of CD4+ T cells independent of the PD-1 receptor. J Exp Med 198, 31-38.
Stephens, R., and J. Langhorne. 2010. Effector memory Th1 CD4 T cells are maintained in a mouse model of chronic malaria. PLoS Pathog 6:e1001208.
Su, Z., and Stevenson, M. M. 2002. IL-12 is required for antibody-mediated protective immunity against blood-stage Plasmodium chabaudi AS malaria infection in mice. J Immunol 168, 1348-1355.
Tran, T. M., S. Li, S. Doumbo, D. Doumtabe, C. Y. Huang, S. Dia, A. Bathily, J. Sangala, Y. Kone, A. Traore, M. Niangaly, C. Dara, K. Kayentao, A. Ongoiba, O. K. Doumbo, B.
Traore, and P. D. Crompton. 2013. An Intensive Longitudinal Cohort Study of Malian Children and Adults Reveals No Evidence of Acquired Immunity to Plasmodium falciparum Infection. Clin Infect Dis 57:40-47.
Tseng, S. Y., M. Otsuji, K. Gorski, X. Huang, J. E. Slansky, S. I. Pai, A. Shalabi, T. Shin, D. M. Pardoll, and H. Tsuchiya. 2001. B7-DC, a new dendritic cell molecule with potent costimulatory properties for T cells. J Exp Med 193:839-846.
Urban, B. C., D. J. Ferguson, A. Pain, N. Willcox, M. Plebanski, J. M. Austyn, and D. J. Roberts. 1999. Plasmodium falciparum-infected erythrocytes modulate the maturation of dendritic cells [see comments]. Nature 400:73-77.
Urban, B. C., N. Willcox, and D. J. Roberts. 2001. A role for CD36 in the regulation of dendritic cell function. Proc Natl Acad Sci USA 98:8750-8755.
Wang et al, 2006, Transcription factor T-bet regulates inflammatory arthritis through its function in dendritic cells, JCI February; 116(2) 414-421
Weiss, G. E., B. Traore, K. Kayentao, A. Ongoiba, S. Doumbo, D. Doumtabe, Y. Kone, S. Dia, A. Guindo, A. Traore, C. Y. Huang, K. Miura, M. Mircetic, S. Li, A. Baughman, D. L. Narum, L. H. Miller, O. K. Doumbo, S. K. Pierce, and P. D. Crompton. 2010. The Plasmodium falciparum-specific human memory B cell compartment expands gradually with repeated malaria infections. PLoS Pathog 6:e1000912.
Wipasa, J., C. Suphavilai, L. C. Okell, J. Cook, P. H. Corran, K. Thaikla, W. Liewsaree, E. M. Riley, and J. C. Hafalla. 2010. Long-lived antibody and B Cell memory responses to the human malaria parasites, Plasmodium falciparum and Plasmodium vivax. PLoS Pathog 6:e1000770.
Wykes, M. N., and M. F. Good. 2008. What really happens to dendritic cells during malaria? Nat Rev Microbiol 6:864-867.
Wykes, M. N., X. Q. Liu, L. Beattie, D. I. Stanisic, K. J. Stacey, M. J. Smyth, R. Thomas, and M. F. Good. 2007a. Plasmodium Strain Determines Dendritic Cell Function Essential for Survival from Malaria. PLoS Pathog 3:e96.
Wykes, M. N., X. Q. Liu, S. Jiang, C. Hirunpetcharat, and M. F. Good. 2007b. Systemic tumor necrosis factor generated during lethal Plasmodium infections impairs dendritic cell function. J Immunol 179:3982-3987.
Zhu, C., Anderson, A. C., Schubart, A., Xiong, H., Imitola, J., Khoury, S. J., Zheng, X. X., Strom, T. B., and Kuchroo, V. K. (2005). The Tim-3 ligand galectin-9 negatively regulates T helper type 1 immunity. Nat Immunol 6, 1245-1252.

1. A method for promoting or enhancing immunity in a mammal, comprising administering a Galectin-9 agonist to the mammal to thereby activate or stimulate Galectin-9 activity in the mammal, wherein the Galectin 9-agonist is multimeric soluble PD-L2 and wherein the multimeric soluble PD-L2 comprises at least 3 PD-L2 monomers.

2. The method of claim 1, wherein the multimeric soluble PD-L2 comprises 3, 4, 5, 6, 7 or 8 PD-L2 monomers.

3. The method of claim 1, wherein the multimeric soluble PD-L2 comprises 8 PD-L2 monomers.

4. The method of claim 1, which treats a disease, disorder or condition in the mammal.

5. The method of claim 1, wherein the mammal is a human.

6. A method for treating a cancer or a disease, disorder or condition caused by a pathogen, in a mammal, comprising administering to the mammal a Galectin-9 agonist, wherein the Galectin 9-agonist is multimeric soluble PD-L2, and wherein the multimeric soluble PD-L2 comprises at least 3 PD-L2 monomers.

7. The method of claim 6, wherein the multimeric soluble PD-L2 comprises 3, 4, 5, 6, 7 or 8 PD-L2 monomers.

8. The method of claim 6, wherein the multimeric soluble PD-L2 comprises 8 PD-L2 monomers.

9. The method of claim 6, wherein the mammal is a human.
